(19) United States
(12) Patent Application Publication
(10) Pub. No.: US 2013/0097734 A1
(43) Pub. Date: Apr. 18, 2013

(54) LATE BLIGHT RESISTANCE GENES

(71) Applicants: Sophien Kamoun, Norwich (GB); Maria Eugenia Segretin, Buenos Aires (AR); Sebastian Schornack, Norwich (GB)

(72) Inventors: Sophien Kamoun, Norwich (GB); Maria Eugenia Segretin, Buenos Aires (AR); Sebastian Schornack, Norwich (GB)

(73) Assignee: TWO BLADES FOUNDATION, Evanston, IL (US)

(21) Appl. No.: 13/547,198

(22) Filed: Jul. 12, 2012

Related U.S. Application Data
(60) Provisional application No. 61/506,829, filed on Jul. 12, 2011.

(51) Int. Cl. C12N 15/82

(52) U.S. Cl. CPC C12N 15/8282
USPC 800/301; 536/23.6; 435/320.1; 435/418; 435/6.11; 435/4; 506/10; 800/279; 530/350

(57) ABSTRACT

Nucleic acid molecules that confer to a plant resistance to the plant pathogenic Phytophthora species are provided. These nucleic acid molecules can be introduced into plants that are otherwise susceptible to infection by certain strains of Phytophthora infestans or other Phytophthora species in order to enhance the resistance of the plant to this plant pathogen. Also provided are the resistance proteins encoded by these nucleic acid molecules. Methods of making nucleic acid molecules that confer upon a plant resistance to a plant pathogen, the nucleic acid molecules made by these methods, the resistance proteins encoded thereby, and methods of using these nucleic acid molecules to increase the resistance of plants to pathogens are further provided.

### CROSS-REFERENCE TO RELATED APPLICATION

This application claims the benefit of U.S. Provisional Patent Application No. 61/506,829, filed Jul. 12, 2011, herein incorporated by reference in its entirety.

### REFERENCE TO A SEQUENCE LISTING SUBMITTED AS A TEXT FILE VIA EFS WEB

The official copy of the sequence listing is submitted electronically via EFS-Web as an ASCII formatted sequence listing with a file named 422128SEQLIST.TXT, created on Jul. 12, 2012, and having a size of 760 kilobytes, and is filed concurrently with the specification. The sequence listing contained in this ASCII formatted document is part of the specification and is herein incorporated by reference in its entirety.

### BACKGROUND OF THE INVENTION

Plants are hosts to thousands of infectious diseases caused by a vast array of phytopathogenic fungi, bacteria, viruses, oomycetes, and nematodes. Plants recognize and resist many invading phytopathogens by inducing a rapid defense response. Recognition is often due to the interaction between a dominant or semi-dominant resistance (R) gene product in the plant and a corresponding dominant avirulence (Avr) gene product expressed by the invading phytopathogen. R-gene triggered resistance often results in a programmed cell-death, that has been termed the hypersensitive response (HR). The HR is believed to constrain spread of the pathogen.

How R gene products mediate perception of the corresponding Avr proteins is mostly unclear. It has been proposed that phytopathogen Avr products function as ligands, and that plant R products function as receptors. In this receptor-ligand model binding of the Avr product to a corresponding R product in the plant initiates the chain of events within the plant that produces HR leads to disease resistance. In an alternate model the R protein perceives the action rather than the structure of the Avr protein. In this model the Avr protein is believed to modify a plant target protein (pathogenicity target) in order to promote pathogen virulence. The modification of the pathogenicity protein is detected by the matching R protein and triggers a defense response. Experimental evidence suggests that some R proteins act as Avr receptors while others detect the activity of the Avr protein.

The production of transgenic plants carrying a heterologous gene sequence is now routinely practiced by plant molecular biologists. Methods for incorporating an isolated gene sequence into an expression cassette, producing plant transformation vectors, and transforming many types of plants are well known. Examples of the production of transgenic plants having modified characteristics as a result of the introduction of a heterologous transgene include: U.S. Pat. No. 5,719,046 to Guerineau (production of herbicide resistant plants by introduction of bacterial dihydropteroate synthase gene); U.S. Pat. No. 5,231,020 to Jorgensen (modification of flavenoids in plants); U.S. Pat. No. 5,583,021 to Dougherty (production of virus resistant plants); and U.S. Pat. No. 5,767,372 to De Greve and U.S. Pat. No. 5,500,365 to Fischoff (production of insect resistant plants by introducing Bacillus thuringiensis genes).

In conjunction with such techniques, the isolation of plant R genes has similarly permitted the production of plants having enhanced resistance to certain pathogens. Since the cloning of the first R gene, Pto from tomato, which confers resistance to Pseudomonas syringae pv. tomato (Martin et al. (1993) Science 262: 1432-1436), a number of other R genes have been reported (Hammond-Kosack & Jones (1997) Ann. Rev. Plant Physiol. Plant Mol. Biol. 48:575-607). A number of these genes have been used to introduce the encoded resistance characteristic into plant lines that were previously susceptible to the corresponding pathogen. For example, U.S. Pat. No. 5,571,706 describes the introduction of the N gene into tobacco lines that are susceptible to Tobacco Mosaic Virus (TMV) in order to produce TMV-resistant tobacco plants. WO 95/28423 describes the creation of transgenic plants carrying the Rps2 gene from Arabidopsis thaliana, as a means of creating resistance to bacterial pathogens including Pseudomonas syringae, and WO 98/02545 describes the introduction of the Prf gene into plants to obtain broad-spectrum pathogen resistance. More recently, the Bs2 and Bs3 genes from pepper, which confer resistance to bacterial spot disease caused by the phytopathogenic bacterium Xanthomonas campestris pv. vesicatoria (Xcv), have been isolated and sequenced, and transgenic plants expressing these genes have been shown to produce a hypersensitive response when challenged with the strains of Xcv expressing the corresponding avirulence genes (U.S. Pat. No. 6,262,343; U.S. Pat. Pub. No. 2009/0133158).

Late blight is one of the most devastating diseases affecting potato (Solanum tuberosum) production worldwide. This disease is caused by the oomycete plant pathogen, Phytophthora infestans. To combat this plant pathogen, potato breeders have introduced at least 11 late blight resistance (R) alleles from Solanum demissum into the cultivated potato (Gebhardt and Valkonen (2001) Annu. Rev. Phytopathol. 39:79-102). The products of R alleles recognize the products of corresponding Avr alleles in races of P. infestans, triggering disease resistance and HR. Recently, the first Avr gene (Avr3a) was identified in P. infestans (Armstrong et al. (2005) PNAS 102:7766-7771). R3a, a resistance protein discovered in potato, can trigger a hypersentive response response upon the recognition of the avirulence effector AVR3a^{KI} from P. infestans but cannot recognize AVR3a^{EM}, the product of another allele that is predominant in pathogen populations. To date, all the characterized P. infestans strains in nature produce at least one of these AVR3a proteins.

### BRIEF SUMMARY OF THE INVENTION

The present invention provides nucleic acid molecules for resistance (R) genes that are modified versions of the R3a resistance gene of potato (Solanum tuberosum L.). The R3a resistance gene is known to confer upon a plant resistance to strains of the oomycte pathogen, Phytophthora infestans, that produce the AVR3a^{KI} effector protein. The R genes of the present invention encode modified R3a resistance proteins, which display altered specificity for effector proteins from Phytophthora infestans and which are capable of causing a hypersensitive response in a plant when expressed in a plant in the presence of a Phytophthora infestans strain that produces the AVR3a^{EM} effector protein. Thus, in one embodiment, the present invention provides nucleic acid molecules comprising a nucleotide sequence encoding a modified R3a protein that is capable of inducing a hypersensitive response in a plant in the presence of AVR3a^{EM}. Preferably, the modified R3a proteins encoded by such nucleic acid molecules also retain the function of the wild-type R3a protein of inducing a hypersensitive response in a plant in the presence of AVR3a^{KI}.

The present invention further provides plants comprising in their genomes one or more heterologous polynucleotides of the invention. The heterologous polynucleotides of the invention comprise a nucleotide sequence encoding a modified R3a protein of the invention and can further comprise an operably linked to promoter capable of driving expression of the nucleotide sequence in a plant. The modified R3a proteins of the invention are capable of inducing a hypersensitive response in a plant in the presence of AVR3a^{EM} and are encoded the nucleic acid molecules of the invention. Preferably, the modified R3a proteins are also capable of inducing a hypersensitive response in a plant in the presence of AVR3a^{KI}.

The present invention provides methods for enhancing the resistance of a plant to Phytophthora infestans. In one embodiment, the methods involve transforming a plant cell, particularly a potato or tomato cell, with a polynucleotide comprising a nucleotide sequence encoding a modified R3a protein of the invention, wherein the modified R3a protein is capable of inducing a hypersensitive response in a plant in the presence of AVR3a^{EM} and preferably is also capable of inducing a hypersensitive response in a plant in the presence of AVR3a^{KI}. The methods can further involve regenerating a transformed plant from the transformed cell, wherein the transformed plant comprises enhanced resistance to at least one strain of Phytophthora infestans.

In another embodiment, the methods for enhancing the resistance of a plant to Phytophthora infestans of the present invention involve enhancing the resistance of a potato plant to Phytophthora infestans. Such methods comprise altering the coding sequence of the R3a gene in a potato plant or cell, whereby the altered coding sequence encodes a modified R3a protein of the invention that comprises an amino acid sequence having at least one amino acid substitution relative to the amino acid sequence of the R3a protein encoded by the R3a gene, wherein the modified R3a protein is capable of inducing a hypersensitive response in a plant in the presence of AVR3a^{EM}. Preferably, the modified R3a protein is also capable of inducing a hypersensitive response in a plant in the presence of AVR3a^{KI}. The coding sequence can, for example, be modified in vivo by targeted mutagenesis, homologous recombination, or mutation breeding. The methods can further involve regenerating a potato plant from the potato cell, wherein the regenerated potato plant comprises enhanced resistance to at least one strain of Phytophthora infestans.

The present invention additionally provides methods of selecting a potato plant for enhanced resistance to Phytophthora infestans. The methods involve screening one or more potato plants or parts or cells thereof either for a nucleotide sequence encoding a modified R3a protein or for a modified R3a protein, wherein the modified R3a protein is capable of inducing a hypersensitive response in a plant in the presence of AVR3a^{EM}, and selecting a potato plant comprising the nucleotide sequence encoding a modified R3a protein or the modified R3a protein.

The present invention further provides methods for making R proteins with altered recognition specificity for an effector protein of a plant pathogen. The methods comprise substituting at least one amino acid in the amino sequence of an R protein with a different amino acid, so as to produce a modified R protein. Prior to being modified, the R protein is capable of causing a hypersensitive response when the unmodified R protein is present in a plant with a first effector protein but is not capable of causing a hypersensitive response when the unmodified R protein is present in a plant with a second effector protein. The modified R protein is capable of causing a hypersensitive response when the modified R protein is present in a plant with the second effector protein and preferably is also capable of hypersensitive response when the modified R protein is present in a plant with the first effector protein. Typically, the methods involve altering the coding sequence of the R protein, whereby the altered coding sequence encodes an amino acid sequence that comprises at least one amino acid substitution when compared to the amino acid sequence of the unmodified R protein. The coding sequence can be altered, for example, by making a targeted change in one or more nucleotides in the coding sequence or by random mutagenesis. In one embodiment of the invention, the R protein is potato R3a protein and the plant pathogen is Phytophthora infestans.

The present invention additionally provides methods for making a modified R protein that is capable of causing in a plant a hypersensitive response of increased severity. The methods comprise substituting at least one amino acid in the amino sequence of an R protein with a different amino acid so as to produce a modified R protein. The modified R protein is capable of causing a hypersensitive response in a plant in the presence of an effector protein, wherein the hypersensitive response is of increased severity, when compared to a hypersensitive response caused in a plant by the unmodified R protein in the presence of the effector protein. Typically, the methods involve altering the coding sequence of the R protein, whereby the altered coding sequence encodes an amino acid sequence that comprises at least one amino acid substitution when compared to the amino acid sequence of the unmodified R protein. The coding sequence can be altered, for example, by making a targeted change in one or more nucleotides in the coding sequence or by random mutagenesis.

Additionally provided are plants, plant parts, seeds, plant cells, other non-human host cells, and expression cassettes comprising one or more of the nucleic acid molecules of the present invention and the R proteins or polypeptides encoded by the coding sequences of the present invention.

The present invention further provides isolated polypeptides comprising AVR3a homologs from Phytophthora palmivora, nucleic acid molecules encoding such AVR3a homologs, and methods of using such polypeptides and nucleic acid molecules. Additionally provided are expression cassettes, bacterial cells, plant cells, and other non-human host cells, plants, plant parts, and seeds, comprising nucleic acid molecules encoding the AVR3a homologs of the present invention.

### BRIEF DESCRIPTION OF THE DRAWINGS

FIG. 1. Artificial evolution to extend R3a recognition specificity: experimental design.

FIG. 2. Co-expression of R3a wild-type and mutant clones with pGR106-AVR3aEM, pGR106-ΔGFP and pGR106-AVR3aKI. 10-12 spots from different plants were coinfiltrated with the mentioned cultures and HR-like phenotypes were scored during 7 d.p.i. The scores plotted represent the mean values at 7 d.p.i. of all the infiltrated spots. HR index is measured in arbitrary units according the characteristics observed: from no visible HR phenotype to confluent necrosis.

FIG. 3. Right Panel: Sequence analysis of selected mutant clones. Left Panel: Details of the mutations for each clone. Mutations is indicated as follows: XnumberY, with X being the original amino acid in R3a at amino acid position (number), and with Y being the amino acid present in the mutant R3a clone.

FIG. 4. Dissection of the contribution of the different mutated sites on R3a to the enhanced recognition of AVR3a^{EM} effector allele. FIG. 4A: chimerical clones between wild-type R3a and clone 1B/A10 were made to separate mutations affecting the CC and NBS domains from that affecting the leucine-rich repeat (LRR) domain. The chimerical clones were transformed into Agrobacterium tumefaciens and used for infiltration assays. FIGS. 4B and 4C show the co-infiltration assays scheme. The denoted closes in FIG. 4B were co-infiltrated with pGR106-AVR3a^{EM}, pGR106-ΔGFP, or pGR106-AVR3a^{KI} as indicated in FIG. 4C. The HR-like responses were analyzed at 4 d.p.i. under white or UV light. Representative leaves of three replicates are shown in FIG. 4C.

FIG. 5. Co-expression of mutated R3a clones that encode modified R3a proteins with a single amino acid substitution with Phytophthora infestans AVR3a (PiAVR3a). The clones were co-infiltrated with PiAVR3a and different variants of PiAVR3a (pGR106 background) into N. benthamiana leaves. The single amino acid substitution clones recognize not only AVR3a^{EM} but also other variants of AVR3a. The phenotypes (HR) were analyzed under UV light 4 d.p.i. All the GS, the 17+ and Ch7 R3a versions were cloned in the pCBNptII_PTvnt1.1 backbone.

FIG. 6. Co-expression of mutated R3a clones with homologs of PiAVR3a. The modified R3a proteins encoded by the mutated R3a clones recognize not only AVR3a^{EM} from Phytophthora infestans (Pi) but also members of the AVR3a family from other Phytophthora species. Mutated R3a clones (all cloned in the pCB302-3 backbone) were co-infiltrated with different members of the AVR3a family from P. sojae (Ps) and P. capsici (Pc) (pTRBO background) in N. benthamiana. PiAVR2 is an unrelated effector, included as a negative control. The phenotypes (HR) were analyzed under UV light 4 d.p.i.

FIG. 7. Co-expression of mutated R3a clones that encode modified R3a proteins with a single amino acid substitution with homologs of PiAVR3. The modified R3a proteins encoded by the mutated R3a clones recognize not only AVR3a^{EM} from Phytophthora infestans (Pi) but also members of the AVR3a family from other Phytophthora species. Mutated R3a clones (all cloned in the pCBNptII_PTvnt1.1 backbone) were co-infiltrated with different members of the AVR3a family from P. sojae (Ps) and P. capsici (Pc) (pTRBO background) in N. benthamiana. Phenotype (HR) was analyzed under UV light 4 d.p.i.

FIG. 8. The modified R3a encoded by the GS4 clone is more sensitive for PiAVR3a^{KI} recognition than is R3a. Several R3a modified clones (GS4, 8, 12 and 15; 6C/C10 and Ch7) or the R3a (wild-type) clone were co-infiltrated side-by-side with serial dilutions of PiAVR3a^{KI} (pK7 backbone) in N. benthamiana as described in Example 3. Phenotype (HR) was scored in one of three categories at 4 d.p.i. for neighboring spots one the same leaf (i.e., modified R3a and R3a in opposite sides of the leaf) as follows: (1) modified R3a stronger than R3a, (2) modified R3a equal to R3a, or (3) modified R3a weaker than R3a. FIG. 8A is a graphical representation of the results for each of the clones expressing a modified R3a protein and empty vector (EV) control. “R3a+” is a modified R3a and “R3a” is wild-type R3a. FIG. 8B is a photograph of a representative N. benthamiana leaf in which the R3a clone was co-infiltrated with a clone expressing PiAVR3a^{KI} on the left side of the leaf and the modified R3a clone (GS4) was co-infiltrated with a clone expressing PiAVR3a^{KI} on the left side of the leaf.

FIG. 9 is a graphical representation of the hypersensitive response in N. benthamiana in the presence of PiAVR3a^{EM} when the modified R3a protein encoded by the GS4 clone is co-infiltrated with a clone encoding R3a (wild-type). HR index is measured in arbitrary units according the characteristics observed: from no visible HR phenotype to confluent necrosis. The HR index was determined at 2.5, 3.5, 4.5, and 5.5 d.p.i. The four lines in the figure from top to bottom represent results from the co-infiltration of clones expressing: (1) R3a, e.v. (empty vector control), and PiAVR3a^{KI}; (2) GS4, e.v., and PiAVR3a^{EM}; (3) R3a, GS4, and PiAVR3a^{EM}; and (4) R3a, e.v., and PiAVR3a^{EM}.

FIG. 10 is a graphical representation of the hypersensitive response in N. benthamiana in the presence of PiAVR3a^{EM} when the modified R3a protein encoded by the GS12 clone is co-infiltrated with a clone encoding R3a (wild-type). HR index is measured in arbitrary units according the characteristics observed: from no visible HR phenotype to confluent necrosis. The HR index was determined at 2.5, 3.5, 4.5, and 5.5 d.p.i. The four lines in the figure from top to bottom represent results from the co-infiltration of clones expressing: (1) R3a, e.v. (empty vector control), and PiAVR3a^{KI}; (2) GS12, e.v., and PiAVR3a^{EM}; (3) R3a, GS12, and PiAVR3a^{EM}; and (4) R3a, e.v., and PiAVR3a^{EM}.

FIG. 11. Infection of R3a transgenic or Wild-type N. benthamiana with a RFP fluorescent P. palmivora 6390 at 3 d.p.i.

FIG. 12. R3a can trigger cell death in non-solanaceous unrelated species. Co-expression of R3a and Avr3a from P. infestans in lamb's lettuce and spinach and visualization of HR/cell death using UV illumination.

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth a nucleotide sequence encoding the wild-type R3a protein.

SEQ ID NO: 2 sets forth the amino acid sequence of the wild-type R3a protein.

SEQ ID NO: 3 sets forth a nucleotide sequence encoding the modified R3a protein corresponding to clone 1A/1A (also referred to herein as 1+).

SEQ ID NO: 4 sets forth the amino acid sequence of the modified R3a protein corresponding to clone 1A/1A.

SEQ ID NO: 5 sets forth a nucleotide sequence encoding the modified R3a protein corresponding to clone 1B/A10 (also referred to herein as 2+).

SEQ ID NO: 6 sets forth the amino acid sequence of the modified R3a protein corresponding to clone 1B/A10.

SEQ ID NO: 7 sets forth a nucleotide sequence encoding the modified R3a protein corresponding to clone 1B/F10 (also referred to herein as 3+).

SEQ ID NO: 8 sets forth the amino acid sequence of the modified R3a protein corresponding to clone 1B/F10.

SEQ ID NO: 9 sets forth a nucleotide sequence encoding the modified R3a protein corresponding to clone 1B/H5 (also referred to as 4+).

SEQ ID NO: 10 sets forth the amino acid sequence of the modified R3a protein corresponding to clone 1B/H5.

SEQ ID NO: 11 sets forth a nucleotide sequence encoding the modified R3a protein corresponding to clone 2A/B5 (also referred to herein as 5+).

SEQ ID NO: 12 sets forth the amino acid sequence of the modified R3a protein corresponding to clone 2A/B5.

SEQ ID NO: 13 sets forth a nucleotide sequence encoding the modified R3a protein corresponding to clone 2A/F11 (also referred to herein as 7+).

SEQ ID NO: 14 sets forth the amino acid sequence of the modified R3a protein corresponding to clone 2A/F11.

SEQ ID NO: 15 sets forth a nucleotide sequence encoding the modified R3a protein corresponding to clone 3A/A10 (also referred to herein as 8+).

SEQ ID NO: 16 sets forth the amino acid sequence of the modified R3a protein corresponding to clone 3A/A10.

SEQ ID NO: 17 sets forth a nucleotide sequence encoding the modified R3a protein corresponding to clone 3B/B4 (also referred to herein as 9+).

SEQ ID NO: 18 sets forth the amino acid sequence of the modified R3a protein corresponding to clone 3B/B4.

SEQ ID NO: 19 sets forth a nucleotide sequence encoding the modified R3a protein corresponding to clone 3B/H1 (also referred to herein as 10+).

SEQ ID NO: 20 sets forth the amino acid sequence of the modified R3a protein corresponding to clone 3B/H1.

SEQ ID NO: 21 sets forth a nucleotide sequence encoding the modified R3a protein corresponding to clone 3D/D3 (also referred to herein as 10+).

SEQ ID NO: 22 sets forth the amino acid sequence of the modified R3a protein corresponding to clone 3D/D3.

SEQ ID NO: 23 sets forth a nucleotide sequence encoding the modified R3a protein corresponding to clone 4B/E10 (also referred to herein as 12+).

SEQ ID NO: 24 sets forth the amino acid sequence of the modified R3a protein corresponding to clone 4B/E10.

SEQ ID NO: 25 sets forth a nucleotide sequence encoding the modified R3a protein corresponding to clone 4D/B3 (also referred to herein as 14+).

SEQ ID NO: 26 sets forth the amino acid sequence of the modified R3a protein corresponding to clone 4D/B3.

SEQ ID NO: 27 sets forth a nucleotide sequence encoding the modified R3a protein corresponding to clone 4D/D10 (also referred to herein as 15+).

SEQ ID NO: 28 sets forth the amino acid sequence of the modified R3a protein corresponding to clone 4D/D10.

SEQ ID NO: 29 sets forth a nucleotide sequence encoding the modified R3a protein corresponding to clone 6A/E5 (also referred to herein as 16+).

SEQ ID NO: 30 sets forth the amino acid sequence of the modified R3a protein corresponding to clone 6A/E5.

SEQ ID NO: 31 sets forth a nucleotide sequence encoding the modified R3a protein corresponding to clone 6C/C10 (also referred to herein as 17+).

SEQ ID NO: 32 sets forth the amino acid sequence of the modified R3a protein corresponding to clone 6C/C10.

SEQ ID NO: 33 sets forth a nucleotide sequence encoding the modified R3a protein corresponding to clone 6D/A1 (also referred to herein as 18+).

SEQ ID NO: 34 sets forth the amino acid sequence of the modified R3a protein corresponding to clone 6D/A1.

SEQ ID NO: 35 sets forth a nucleotide sequence encoding the modified R3a protein corresponding to clone 6D/E6 (also referred to herein as 19+).

SEQ ID NO: 36 sets forth the amino acid sequence of the modified R3a protein corresponding to clone 6D/E6.

SEQ ID NO: 37 sets forth a nucleotide sequence of BAC clone SH23G23.

SEQ ID NO: 38 sets forth the nucleotide sequence of primer R3a_BamHI_Fw_MES.

SEQ ID NO: 39 sets forth the nucleotide sequence of primer R3a_SpeI_Rev_MES.

SEQ ID NO: 40 sets forth the nucleotide sequence of the Rpi-vnt1.1 promoter.

SEQ ID NO: 41 sets forth the nucleotide sequence of the Rpi-vnt1.1 terminator.

SEQ ID NO: 42 sets forth a nucleotide sequence encoding the modified R3a protein corresponding to clone GS4.

SEQ ID NO: 43 sets forth the amino acid sequence of the modified R3a protein corresponding to clone GS4.

SEQ ID NO: 44 sets forth a nucleotide sequence encoding the modified R3a protein corresponding to clone GS8.

SEQ ID NO: 45 sets forth the amino acid sequence of the modified R3a protein corresponding to clone GS8.

SEQ ID NO: 46 sets forth a nucleotide sequence encoding the modified R3a protein corresponding to clone GS9.

SEQ ID NO: 47 sets forth the amino acid sequence of the modified R3a protein corresponding to clone GS9.

SEQ ID NO: 48 sets forth a nucleotide sequence encoding the modified R3a protein corresponding to clone GS12.

SEQ ID NO: 49 sets forth the amino acid sequence of the modified R3a protein corresponding to clone GS12.

SEQ ID NO: 50 sets forth a nucleotide sequence encoding the modified R3a protein corresponding to clone GS15.

SEQ ID NO: 51 sets forth the amino acid sequence of the modified R3a protein corresponding to clone GS15.

SEQ ID NO: 52 sets forth a nucleotide sequence encoding the modified R3a protein corresponding to clone CT* (also referred to has Ch7 or 2+Ch7).

SEQ ID NO: 53 sets forth the amino acid sequence of the modified R3a protein corresponding to clone CT*.

SEQ ID NO: 54 sets forth the amino acid sequence of an AVR3a homolog from Phytophthora palmivora designated as AJ1A.

SEQ ID NO: 55 sets forth the amino acid sequence of an AVR3a homolog from Phytophthora palmivora designated as AJ1B.

SEQ ID NO: 56 sets forth the amino acid sequence of an AVR3a homolog from Phytophthora palmivora designated as AJ2A.

SEQ ID NO: 57 sets forth the amino acid sequence of an AVR3a homolog from Phytophthora palmivora designated as AJ3A.

SEQ ID NO: 58 sets forth the amino acid sequence of an AVR3a homolog from Phytophthora palmivora designated as AJ4A.

SEQ ID NO: 59 sets forth the amino acid sequence of an AVR3a homolog from Phytophthora palmivora designated as AJ5A.

SEQ ID NO: 60 sets forth the amino acid sequence of an AVR3a homolog from Phytophthora palmivora designated as L1B.

SEQ ID NO: 61 sets forth the amino acid sequence of an AVR3a homolog from Phytophthora palmivora designated as L2A.

SEQ ID NO: 62 sets forth the amino acid sequence of an AVR3a homolog from Phytophthora palmivora designated as L3A.

SEQ ID NO: 63 sets forth the amino acid sequence of an AVR3a homolog from Phytophthora palmivora designated as L3B.

SEQ ID NO: 64 sets forth the amino acid sequence of an AVR3a homolog from Phytophthora palmivora designated as L4A.

SEQ ID NO: 65 sets forth the amino acid sequence of an AVR3a homolog from Phytophthora palmivora designated as LSA.

SEQ ID NO: 66 sets forth the amino acid sequence of an AVR3a homolog from Phytophthora palmivora designated as L6A.

SEQ ID NO: 67 sets forth the amino acid sequence of an AVR3a homolog from Phytophthora palmivora designated as L6B.

SEQ ID NO: 68 sets forth the amino acid sequence of an AVR3a homolog from Phytophthora palmivora designated as L7A.

SEQ ID NO: 69 sets forth the amino acid sequence of an AVR3a homolog from Phytophthora palmivora designated as NODE_{—}55578.

SEQ ID NO: 70 sets forth the amino acid sequence of an AVR3a homolog from Phytophthora palmivora designated as NODE_{—}238692.

SEQ ID NO: 71 sets forth the amino acid sequence of an AVR3a homolog from Phytophthora palmivora designated as NODE_{—}248107.

SEQ ID NO: 72 sets forth the amino acid sequence of an AVR3a homolog from Phytophthora palmivora designated as NODE_{—}279538.

SEQ ID NO: 73 sets forth the amino acid sequence of an AVR3a homolog from Phytophthora palmivora designated as NODE_{—}156862.

SEQ ID NO: 74 sets forth the amino acid sequence of an AVR3a homolog from Phytophthora palmivora designated as AJ2B.

SEQ ID NO: 75 sets forth the amino acid sequence of an AVR3a homolog from Phytophthora palmivora designated as AJ3B.

SEQ ID NO: 76 sets forth the amino acid sequence of an AVR3a homolog from Phytophthora palmivora designated as AJ6B.

SEQ ID NO: 77 sets forth the amino acid sequence of an AVR3a homolog from Phytophthora palmivora designated as AJ7A.

SEQ ID NO: 78 sets forth the amino acid sequence of an AVR3a homolog from Phytophthora palmivora designated as AJ8A.

SEQ ID NO: 79 sets forth the amino acid sequence of an AVR3a homolog from Phytophthora palmivora designated as L1A.

SEQ ID NO: 80 sets forth the amino acid sequence of an AVR3a homolog from Phytophthora palmivora designated as L3C.

SEQ ID NO: 81 sets forth the amino acid sequence of an AVR3a homolog from Phytophthora palmivora designated as L4C.

SEQ ID NO: 82 sets forth the amino acid sequence of an AVR3a homolog from Phytophthora palmivora designated as L6C.

SEQ ID NO: 83 sets forth the amino acid sequence of an AVR3a homolog from Phytophthora palmivora designated as L7B.

SEQ ID NO: 84 sets forth the amino acid sequence of an AVR3a homolog from Phytophthora palmivora designated as L7C.

### DETAILED DESCRIPTION OF THE INVENTION

In the context of this disclosure, a number of terms and abbreviations are used. The following definitions are provided.

“R protein” and “R gene product” are equivalent terms that can be used interchangeably herein and that refer to the gene product of a plant resistance gene referred to an “R gene”. For the present invention, such an R protein or R gene product is a protein that, when expressed in a plant, particularly at the site of infection of a pathogen, is capable of initiating a hypersensitive response (HR) which is characterized by a programmed cell death response in the immediate vicinity of the pathogen. The methods of the present invention do not depend on the use of particular coding sequence for an R protein or R gene product. Any coding sequence of any R gene product can be employed in methods disclosed herein, including, for example, the coding sequences of the R3a protein and of the modified R3a proteins of the invention.

“Modified R protein”, “modified R gene product”, “mutant R protein”, and “mutant R gene product” are equivalent terms that can be used interchangeably herein and that refer to an R protein or R gene product has at least one amino acid substitution when compared to another R protein. Preferably, the modified R proteins of the present invention comprise an amino acid sequence comprising at least one amino acid substitution when compared to an R protein prior to being modified or altered by the methods disclosed herein or any other methods known in the art for modifying the amino acid sequence of a protein. In certain embodiments of the invention, the R protein that is modified or altered by the methods disclosed herein is a native R protein found in a plant including both wild-type R proteins, naturally occurring, mutant R proteins, and other allelic forms. In other embodiments of the invention, the R protein that is modified or altered by the methods disclosed herein was previously modified by methods of the present invention or by any other method known in the art.

“Modified R gene”, “modified R polynucleotide”, “mutant R gene”, and “mutant R polynucleotide” are equivalent terms that can be used interchangeably herein and that refer to gene or polynucleotide that encodes a modified R protein of the invention or fragment thereof.

By “heterologous polynucleotide” is intended a polynucleotide that is not native or endogenous to the genome of a plant, other organism, or host cell. Such heterologous polynucleotides include, for example, any nucleic acid molecules or polynucleotides that are introduced into the genome of a plant as disclosed herein and further include native or endogenous genes that are modified in vivo or in planta as disclosed hereinbelow by methods known in the art such as, for example, targeted mutagenesis, homologous recombination, and mutation breeding.

The present invention is based on the discovery that a plant resistance (R) protein that is specific to an oomcyte plant pathogen can be modified to alter the recognition specificity of the R protein. As is described in detail hereinebelow, a random mutagenesis approach was employed to make modified versions of the potato R3a protein, a resistance protein that is encoded by the R3a gene. The R3a gene is known to confer upon a plant resistance to strains of the oomycte pathogen, Phytophthora infestans, that produce the AVR3a^{KI} effector protein. The resistance mechanism involves a hypersensitive response in the host plant comprising the R3a protein. The hypersensitive response is initiated by recognition of the P. infestans avirulence effector AVR3a^{KI} by the R3a protein in the host plant. However, in pathogen populations of P. infestans, the effector AVR3a^{EM} predominates, and the R3a protein does not recognize AVR3a^{EM} and initiate a hypersensitive response in a plant. Therefore, the R3a resistance gene does not provide resistance against P. infestans strains that produce AVR3a^{EM} but do not produce AVR3a^{KI}. The discovery that led to the present invention is that modified R3a proteins comprising one or more amino acid substitutions relative the wild-type R3a amino sequence can initiate in a plant a hypersensitive response in the presence of AVR3a^{EM}. Moreover, these modified R3a proteins retain the function of initiating in a plant a hypersensitive response in the presence of AVR3a^{KI}. Accordingly, the present invention finds use in enhancing the resistance of crop plants to plant pathogens.

The present invention provides nucleic acid molecules for R genes that are modified versions of the R3a resistance gene of potato (Solanum tuberosum L.). The R genes of the present invention encode modified R3a resistance proteins, which display altered specificity for effector proteins from Phytophthora infestans and which are capable of causing a hypersensitive response in a plant when expressed in a plant in the presence of a Phytophthora infestans strain that produces the AVR3a^{EM} effector protein. Thus, in one embodiment, the present invention provides nucleic acid molecules comprising a nucleotide sequence encoding a modified R3a protein that is capable of inducing a hypersensitive response in a plant in the presence of AVR3a^{EM}. Preferably, the modified R3a proteins encoded by such nucleic acid molecules also retain the function of the wild-type R3a protein of inducing a hypersensitive response in a plant in the presence of AVR3a^{KI}. Such nucleic acid molecules find use in enhancing the resistance of plants to plant pathogens by, for example, the methods of the present invention described hereinbelow.

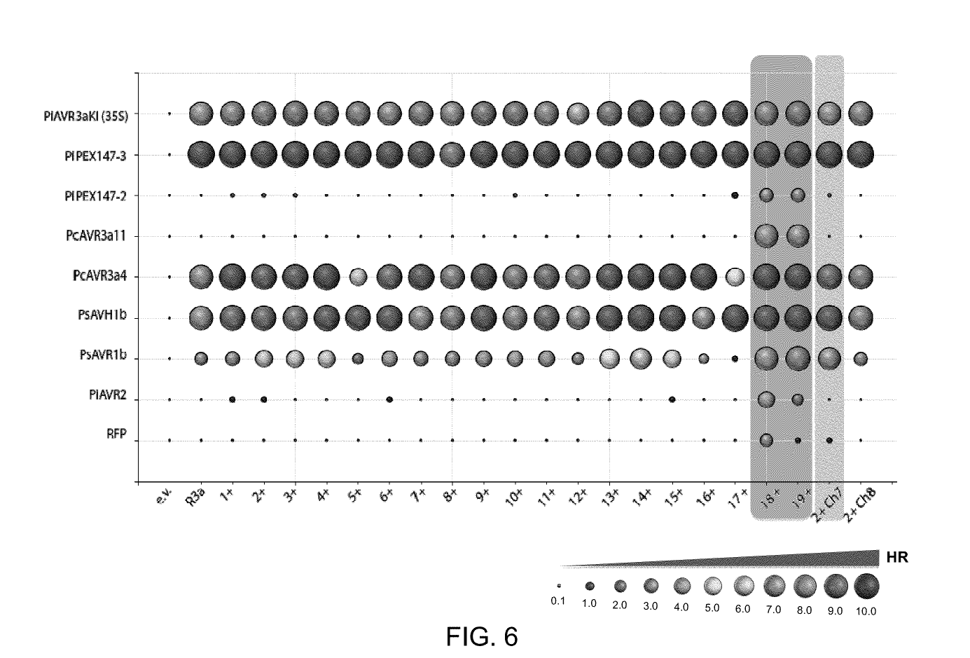

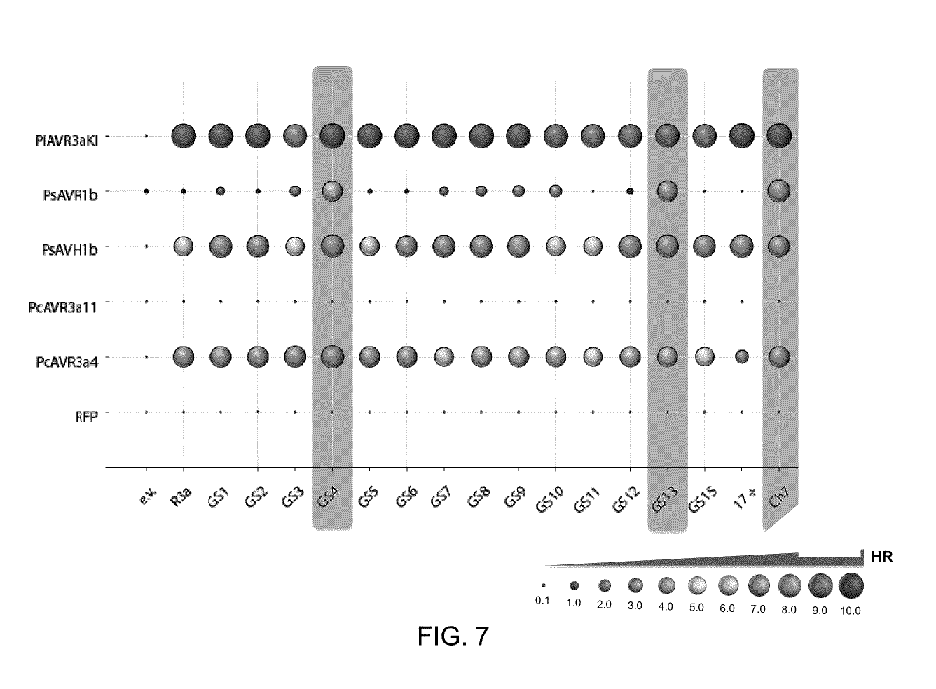

In addition to recognizing AVR3a^{EM} from P. infestans, modified R3a proteins of the present invention have also been found to recognize AVR3a homologs from other Phytophthora species (FIGS. 6-7) including, but not limited to, P. sojae, P. capsici, and P. palmivora. Accordingly, the methods and compositions disclosed herein not only find use in enhancing the resistance of potato and tomato to P. infestans, but also find use in enhancing the resistance of other plant species, particularly monocot and dicot crop plant species, to one or more Phytophthora species such as, for example, P. infestans, P. sojae, P. capsici, and P. palmivora.

In addition to potato and tomato, plants of interest for the present invention include, but are not limited to, pepper (Capsicum spp.), soybean, palms, eggplant (Solanum melongena), petunia (Petunia×hybrida), Physalis sp., woody nightshade (Solanum dulcamara), garden huckleberry (Solanum scabrum), gboma eggplant (Solanum macrocarpon), the asteraceous weeds, Ageratum conyzoides and Solanecio biafrae, and cocoa (Theobroma cacao).

In one aspect, the present invention provides nucleic acid molecules comprising nucleotide sequences encoding modified R proteins, particularly modified R3a proteins. Such nucleic acid molecules find use in methods for expressing the R3a protein in a plant, plant part, plant cell, or other non-human host cell. Non-human host cells of the present invention include, but are not limited to, plant cells, animal cells, bacterial cells, oomycte cells, and fungal cells.

Nucleic acid molecules that comprise nucleotide sequences encoding modified R3a proteins of the present invention include, but are not limited to, nucleic acid molecules comprising: a nucleotide sequences set forth in SEQ ID NO: 3, 5, 7, 9, 11, 13, 15, 17, 19, 21, 23, 25, 27, 29, 31, 33, 35, 42, 44, 46, 48, 50, or 5; or a nucleotide sequence encoding an amino acid sequence set forth in SEQ ID NO: 4, 6, 8, 10, 12, 14, 16, 18, 20, 22, 24, 26, 28, 30, 32, 34, 36, 43, 45, 47, 49, 51, or 53. The nucleotide sequence of the wild-type (i.e., not modified) potato R3a gene is set forth in SEQ ID NO: 1 and the amino acid sequence of the wild-type R3a protein encoded thereby is set forth in SEQ ID NO: 2.

Modified R3a proteins of the present invention include, but are not limited to, polypeptides comprising: an amino acid sequence set forth in SEQ ID NO: 4, 6, 8, 10, 12, 14, 16, 18, 20, 22, 24, 26, 28, 30, 32, 34, 36, 43, 45, 47, 49, 51, or 53; or an amino acid sequence encoded by a nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52.

In one embodiment, the present invention provides nucleic acid molecules encoding modified R3a proteins that comprise an amino acid sequence that differs from the wild-type R3a amino acid sequence by a single amino acid substitution and the modified R3a proteins encoded thereby. Preferably, the single amino acid substitution is in the LRR domain of the R3a protein. More preferably, the single amino acid substitution is selected from the group consisting of L668P, K920E, E941K, C950R, E983K, and K1250R. Nucleic acid molecules encoding such modified R3a proteins nucleic acid molecules include, but are not limited to, nucleic acid molecules comprising: a nucleotide sequence set forth in SEQ ID NO: 31, 42, 44, 48, 50, or 52; or a nucleotide sequence encoding an amino acid sequence set forth in SEQ ID NO: 32, 43, 45, 49, 51, or 53. Such modified R3a proteins include, but are not limited to, polypeptides or proteins comprising: an amino acid sequence set forth in SEQ ID NO: 32, 43, 45, 49, 51, or 53; or an amino acid sequence encoded by a nucleotide sequence set forth in SEQ ID NO: 31, 42, 44, 48, 50 or 52.

For expression of a modified R protein of the present invention in a plant or plant cell, the methods of the invention involve transforming a plant or plant cell with a polynucleotide of the present invention that encodes the modified R protein. Such a nucleotide molecule can be operably linked to a promoter that drives expression in a plant cell. Any promoter known in the art can be used in the methods of the invention including, but not limited to, constitutive promoters, pathogen-inducible promoters, wound-inducible promoters, tissue-preferred promoters, and chemical-regulated promoters. The choice of promoter will depend on the desired timing and location of expression in the transformed plant or other factors. In one embodiment of the invention, the R3a promoter is employed to increase the expression of a modified R3a protein in a plant.

The invention further provides methods for enhancing the resistance of a plant to a plant pathogen, particularly an oomycete plant pathogen, more particularly Phytophthora infestans. The methods comprise transforming a plant cell with a polynucleotide comprising a nucleotide sequence encoding a modified R protein, wherein the modified R protein is capable of inducing a hypersensitive response in a plant in the presence of an effector protein produced by the plant pathogen. Prior to being modified by the methods disclosed herein, the R protein was not capable of initiating in a plant a hypersensitive response in the presence of the effector protein. The methods of the invention can further comprise regenerating the transformed plant cell into a transformed plant.

In one embodiment, the invention provides methods for enhancing the resistance of a plant, particularly a potato or tomato plant, to Phytophthora infestans. The methods comprise transforming a plant cell with a polynucleotide comprising a nucleotide sequence encoding a modified R3a protein of the invention, wherein the modified R3a protein is capable of inducing a hypersensitive response in a plant in the presence of AVR3a^{EM} and preferably is also capable of inducing a hypersensitive response in a plant in the presence of AVR3a^{KI}. Nucleotide sequences encoding modified R3a proteins of the invention that can be used in the methods disclosed herein include, but are not limited to, the nucleotide sequences set forth in SEQ ID NOS: 3, 5, 7, 9, 11, 13, 15, 17, 19, 21, 23, 25, 27, 29, 31, 33, 35, 42, 44, 46, 48, 50, and 52 and fragments and variants thereof that encode modified R3a proteins that that are able to initiate in a plant a hypersensitive response in the presence of at least one effector protein that also is recognized by the full-length, modified R3a protein from which the fragment or variant was derived. If desired, the methods can further involve regenerating a transformed plant from the transformed plant cell. Such transformed plants comprise enhanced resistance to at least one strain of Phytophthora infestans, particularly a strain of Phytophthora infestans that produces AVR3a^{EM}.

In another embodiment, the methods for enhancing the resistance of a plant to Phytophthora infestans involve enhancing the resistance of a potato plant to Phytophthora infestans. Such methods comprise altering the coding sequence of the R3a gene in a plant or plant cell, whereby the altered coding sequence encodes a modified R3a protein of the invention that comprises an amino acid sequence having at least one amino acid substitution relative to the amino acid sequence of the R3a protein encoded by the R3a gene, wherein the modified R3a protein is capable of inducing a hypersensitive response in a plant in the presence of AVR3a^{EM}. Preferably, the modified R3a protein is also capable of inducing a hypersensitive response in a plant in the presence of AVR3a^{KI}. The coding sequence can, for example, be altered in vivo or in planta by targeted mutagenesis, homologous recombination, or mutation breeding. The methods can further involve regenerating a transformed plant from the transformed cell, wherein the transformed plant comprises enhanced resistance to at least one strain of Phytophthora infestans.

Any methods known in the art for modifying DNA in the genome of a plant can used to alter the coding sequences of the R3a gene in planta. Such methods include, for example, methods involving targeted mutagenesis, homologous recombination, and mutation breeding. Targeted mutagenesis or similar techniques are disclosed in U.S. Pat. Nos. 5,565,350; 5,731,181; 5,756,325; 5,760,012; 5,795,972 and 5,871,984; all of which are herein incorporated in their entirety by reference. Methods for gene modification or gene replacement involving homologous recombination can involve inducing double breaks in DNA using zinc-finger nucleases or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Arnould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

TAL effector nucleases can also be used to make double-strand breaks at specific recognition sequences in the genome of a plant for gene modification or gene replacement through homologous recombination. TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

Mutation breeding methods can involve, for example, exposing the plants or seeds to a mutagen, particularly a chemical mutagen such as, for example, ethyl methanesulfonate (EMS) and selecting for plants that possess a desired modification in the R3a gene. However, other mutagens can be used in the methods disclosed herein including, but not limited to, radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Further details of mutation breeding can be found in “Principals of Cultivar Development” Fehr, 1993 Macmillan Publishing Company the disclosure of which is incorporated herein by reference.

The present invention further provides methods for making R proteins with altered recognition specificity for an effector protein of a plant pathogen. The proteins produced by these methods find use in enhancing the resistance of plants to plant pathogens by, for example, the methods disclosed herein. The methods comprise substituting at least one amino acid in the amino sequence of an R protein with a different amino acid, so as to produce a modified R protein. Preferably, the R protein is an R protein that initiates in a plant a hypersensitive response in the presence of an effector protein from an oomycte plant pathogen. More preferably, the R protein is an R protein from potato or tomato that initiates in a plant a hypersensitive response in the presence of an effector protein from the oomycete plant pathogen, Pythophthora infestans. Most preferably, the R protein is the R3a protein.

Prior to being altered by the methods of the invention, the R protein is capable of causing a hypersensitive response when the unmodified R protein is present in a plant with a first effector protein from a plant pathogen but is not capable of causing a hypersensitive response when the unmodified R protein is present in a plant with a second effector protein from the plant pathogen. Generally, the first and second effector proteins are from the same species of plant pathogen but may be from different strains or genotypes of the plant pathogen, wherein the first effector protein is from a first strain or genotype of the plant pathogen and the second effector protein is from a second strain or genotype of the plant pathogen.

The modified R protein is capable of causing a hypersensitive response when the modified R protein is present in a plant with the second effector protein and preferably is also capable of hypersensitive response when the modified R protein is present in a plant with the first effector protein. In one embodiment of the invention, the modified R protein is the R protein is a modified potato R3a protein, the first effector is AVR3a^{KI} of Phytophthora infestans, and the second effector is AVR3a^{EM} of Phytophthora infestans.

The methods for making R proteins with altered recognition specificity comprise altering the coding sequence of the R protein, whereby the altered coding sequence encodes an amino acid sequence that comprises at least one amino acid substitution when compared to the amino acid sequence of the unmodified R protein. The coding sequence can be altered, for example, by making a targeted change in one or more nucleotides in the coding sequence (i.e., site directed mutagenesis) or by random mutagenesis. If desired, the altered coding sequences can then used in assays for determining if the protein encoded thereby initiates in a plant a hypersensitive response in the presence of the second effector. Similarly, the altered coding sequences can then used in assays for determining if the protein encoded thereby initiates in a plant a hypersensitive response in the presence of the second effector. The present invention does not depend on particular methods of determining whether the proteins encoded by the altered coding sequences are capable of initiating in a plant a hypersensitive response in the presence of either the first or second effectors.

An example of a preferred method for determining if the protein encoded by an altered coding sequence initiates in a plant a hypersensitive response in the presence of an effector is set forth below in Example 1. This method involves expressing a protein encoded by an altered coding sequence of the invention in a first Agrobacterium tumefaciens culture, expressing an effector protein in a second A. tumefaciens culture, co-infiltrating cells from each of the A. tumefaciens cultures into Nicotiana benthamiana leaves, and then monitoring the leaves after the co-infiltration to determine if hypersensitive response occurred (see, Van der Hoorn et al. (2000) Mol. Plant-Microbe Interact. 13:439-446; Bos et al. (2006) Plant J. 48:165-176; Bos et al. (2009) Mol. Plant-Microbe Interact. 22: 269-281). If desired, the severity of the hypersensitive response can also be evaluated as described in Example 1 below.

In another embodiment, the present invention provides methods for making a modified R protein that is capable of causing in a plant a hypersensitive response of increased severity in the presence of an effector protein of a plant pathogen. The methods comprise altering the coding sequence of the R protein, whereby the altered coding sequence encodes an amino acid sequence that comprises at least one amino acid substitution when compared to the amino acid sequence of the unmodified R protein essentially as described above for the methods for making R proteins with altered recognition specificity. However, in the instant embodiment, the modified R protein is capable of causing a hypersensitive response in a plant in the presence of an effector protein that is of increased severity, when compared to a hypersensitive response caused in a plant by the unmodified R protein in the presence of the effector protein.

If desired, the altered coding sequences can then used in assays for determining if the protein encoded thereby initiates in a plant a hypersensitive response of increased severity in the presence of the effector when compared to the R protein encoded by the unaltered coding sequence. An example of a preferred method for determining if the protein encoded by an altered coding sequence initiates in a plant a hypersensitive response in the presence of an effector, including how to determine the severity of the hypersensitive response, is described above and also set forth below in Example 1.

The methods for enhancing the resistance of a plant to at least one plant pathogen find use in increasing or enhancing the resistance of plants, particularly agricultural or crop plants, to plant pathogens. The methods of the invention can be used with any plant species including monocots and dicots. Preferred plants include Solanaceous plants, such as, for example, potato (Solanum tuberosum), tomato (Lycopersicon esculentum), eggplant (Solanum melongena), pepper (Capsicum spp.; e.g., Capsicum annuum, C. baccatum, C. chinense, C. frutescens, C. pubescens, and the like), tobacco (Nicotiana tabacum, Nicotiana benthamiana), and petunia (Petunia spp., e.g., Petunia×hybrida or Petunia hybrida). Preferred plants of the invention also include any plants that known to be infected by P. infestans or other plant pathogenic Phytophthora species such as, for example, eggplant (Solanum melongena), petunia (Petunia×hybrida), Physalis sp., woody nightshade (Solanum dulcamara), garden huckleberry (Solanum scabrum), gboma eggplant (Solanum macrocarpon), the asteraceous weeds, Ageratum conyzoides and Solanecio biafrae, palms, cocoa (Theobroma cacao), lamb's lettuce (Valerianella locusta), and spinach (Spinacia oleracea).

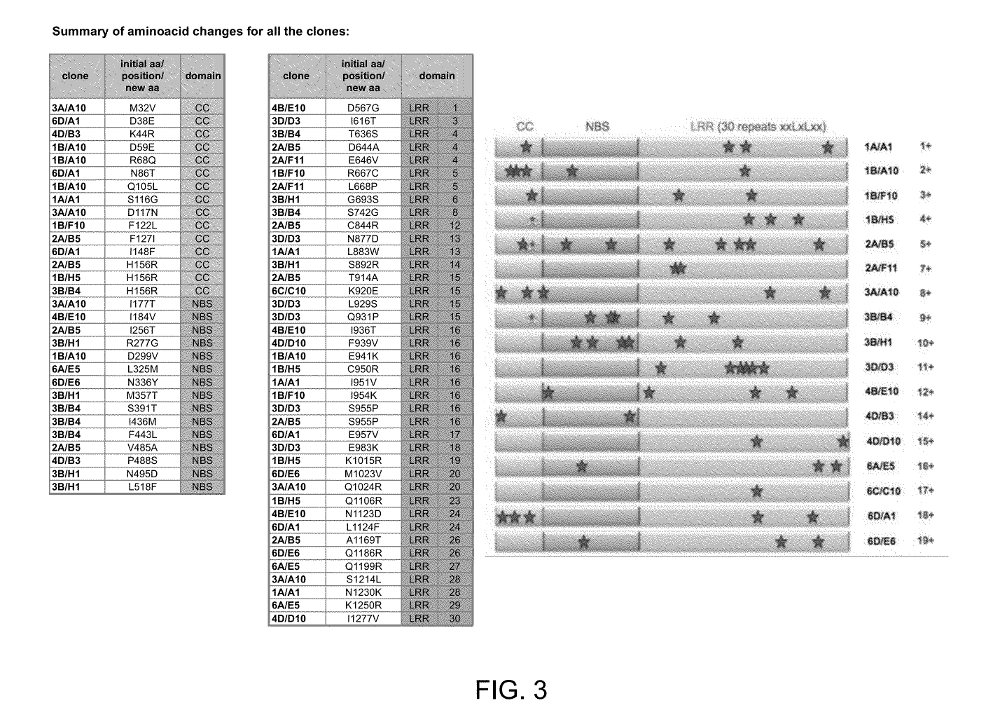

The present invention further provides methods of selecting a potato plant for enhanced resistance to Phytophthora infestans. The methods comprise screening one or more potato plants or parts or cells thereof for nucleotide sequence encoding a modified R3a protein or for a modified R3a protein, wherein the modified R3a protein is capable of inducing a hypersensitive response in a plant in the presence of AVR3a^{EM}, and selecting a potato plant comprising the nucleotide sequence encoding a modified R3a protein or the modified R3a protein. Preferably, the modified R3a protein comprises at least one amino acid substitution as set forth in FIG. 3. More preferably, the modified R3a protein comprises at least one amino acid substitution in the LRR domain as set forth in FIG. 3. In certain embodiments of the invention, the modified R3a protein comprises the amino acid sequence set forth in SEQ ID NO: 4, 6, 8, 10, 12, 14, 16, 18, 20, 22, 24, 26, 28, 30, 32, 34, 36, 43, 45, 47, 49, 51, or 53 or is encoded in the genome of the potato plant by the nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52.

Any methods disclosed herein or otherwise known in the art can be used to screen the one or more potato plants or parts or cells thereof for nucleotide sequence encoding a modified R3a protein including, for example, nucleic acid sequencing and/or methods involving PCR. Likewise, any methods disclosed herein or otherwise known in the art can be used to screen the one or more potato plants or parts or cells thereof for a modified R3a protein, including, for example, amino acid sequencing and immunological methods that discriminate between a wild-type R3a protein and a modified R3a protein.

In certain embodiments of the invention, the potato plants to be screened are from a population of potato plants that are expected to comprise some plants with modified R3a proteins. Such populations can include, for example, populations with naturally occurring genetic variation or populations that comprise induced mutations that are the result of treating potato plants or parts or seeds thereof with, for example, a chemical mutagen or radiation. In other embodiments of the invention, a TILLING (targeting induced local lesions in genomes) population is screened. The use of TILLING populations is disclosed in McCallum et al. (2000) Plant Physiol. 123:439-442; Slade et al. (2005) Nature Biotech. 23:75-81; Oleykowski et al. (1998) Nuc. Acids Res. 26:4597-4602; Neff et al. (1998) Plant J. 14:387-392; all of which are herein incorporated by reference.

It is recognized that potato plants selected for enhanced resistance to Phytophthora infestans by the methods of the present invention find use in breeding potato cultivars with enhanced resistance Phytophthora infestans. Thus, the invention further provides methods of enhancing the resistance of a potato plant to Phytophthora infestans. The methods comprise crossing a first potato plant with a second potato plant, wherein the first potato plant that was selected for enhanced resistance to Phytophthora infestans as disclosed above. Progeny plants resulting from said crossing comprise enhanced resistance to Phytophthora infestans, when compared to the resistance of at least one of the first plant and the second plant. The invention further encompasses the progeny plant and its descendants comprising the enhanced resistance as well as plant parts, plant cells and seeds thereof.

While the present invention does not depend on particular biological mechanism, it is recognized that the R3a protein may act in vivo as dimer and that the presence of an R3a protein and a modified R3a protein of the present invention in the same plant and/or cell may in some situations delay, inhibit, or otherwise negatively affect the triggering of HR by the modified R3a protein in the plant or cell in the presence of AVR3a^{EM}. It is further recognized that in certain embodiments of the invention it may be advantageous to express in a plant, particularly a potato plant, a modified R3a protein at a sufficiently high level to overcome or lessen any negative effect due to the presence of an R3a in the plant or cell thereof, particularly R3a expressed from an endogenous or native R3a gene. In other embodiments, the methods of the present invention can comprise reducing or eliminating the expression of an endogenous or native R3a gene in plant or cell thereof using any method disclosed herein or otherwise known in the art. Such methods of reducing or eliminating the expression of a gene include, for example, in vivo targeted mutagenesis, homologous recombination, and mutation breeding. In one embodiment of the methods of the present invention, the expression of an endogenous or native R3a gene is eliminated in a plant by the replacement of the endogenous or native R3a gene or part thereof with a polynucleotide encoding a modified R3a protein or part thereof through a method involving homologous recombination as described hereinabove. In such an embodiment, the methods can further comprise selfing a heterozygous plant comprising one copy of the polynucleotide and one copy of the endogenous or native R3a gene and selecting for a progeny plant that is homozygous for the polynucleotide.

Nucleic acid molecules of the present invention that comprise nucleotide sequences encoding AVR3a homologs from Phytophthora palmivora include, but are not limited to, nucleic acid molecules encoding an amino acid sequence set forth in SEQ ID NO: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84. Polypeptides of the present invention that are AVR3a homologs from Phytophthora palmivora invention include, but are not limited to, polypeptides comprising: an amino acid sequence set forth in SEQ ID NO: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84. Such nucleic acid molecules and polypeptides find use in the methods disclosed herein for making modified R3a proteins, particularly modified R3a proteins that are capable of inducing a hypersensitive response in a plant in the presence of one or more AVR3a homologs from Phytophthora palmivora. It is recognized that nucleic acid molecules encoding the AVR3a homologs of the present invention can be used in any of the methods disclosed herein which involve the use of nucleic acid molecules encoding AVR3a^{KI} and/or AVR3a^{EM}.

The present invention encompasses isolated or substantially purified polynucleotide (also referred to herein as “nucleic acid molecule”, “nucleic acid” and the like) or protein (also referred to herein as “polypeptide”) compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the full-length or native protein and hence retain the ability to initiate in a plant a hypersensitive response in the presence of a effector protein from a plant pathogen. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

A fragment of a modified R protein that encodes a biologically active portion of a modified R protein of the invention will encode at least 15, 25, 30, 50, 100, 150, 200, 250, 300, 500, 600, 700, 800, 900, 1000, 1100, or 1200 contiguous amino acids, or up to the total number of amino acids present in a full-length, modified R protein of the invention (for example, 1282 amino acids for each of SEQ ID NOS: 4, 6, 8, 10, 12, 14, 16, 18, 20, 22, 24, 26, 28, 30, 32, 34, 36, 43, 45, 47, 49, 51, and 53). Fragments of a modified R polynucleotide that are useful as hybridization probes or PCR primers generally need not encode a biologically active portion of a modified R protein.

Thus, a fragment of a modified R polynucleotide may encode a biologically active portion of a modified R protein, or it may be a fragment that can be used as a hybridization probe or PCR primer using methods disclosed below. A biologically active portion of a modified R protein can be prepared by isolating a portion of one of the modified R polynucleotides of the invention, expressing the encoded portion of the modified R protein (e.g., by recombinant expression in vitro), and assessing the activity of the encoded portion of the modified R protein. Polynucleotides that are fragments of a modified R nucleotide sequence comprise at least 16, 20, 50, 75, 100, 125, 150, 175, 200, 250, 300, 350, 400, 450, 500, 550, 600, 650, 700, 800, 900, 1000, 1100, 1200, 1300, 1400, 1500, 2000, 2500, 3000, or 3500 contiguous nucleotides, or up to the number of nucleotides present in a full-length modified R polynucleotide disclosed herein (for example, 3849 nucleotides for each of SEQ ID NOS: 3, 5, 7, 9, 11, 13, 15, 17, 19, 21, 23, 25, 27, 29, 31, 33, 35, 42, 44, 46, 48, 50, and 52).

A fragment of an AVR3a homolog that encodes a biologically active portion of an AVR3a homolog of the invention will encode at least 15, 25, 30, 50, 75, 100, 110, 120, 130, or 140 contiguous amino acids, or up to the total number of amino acids present in a full-length, AVR3a homolog of the invention. Fragments of an AVR3a homolog that are useful as hybridization probes or PCR primers generally need not encode a biologically active portion of an AVR3a homolog.

Thus, a fragment of an AVR3a homolog may encode a biologically active portion of an AVR3a homolog, or it may be a fragment that can be used as a hybridization probe or PCR primer using methods disclosed below. A biologically active portion of an AVR3a homolog can be prepared by isolating a portion of one of the AVR3a homolog polynucleotides of the invention, expressing the encoded portion of the AVR3a homolog (e.g., by recombinant expression in vitro), and assessing the activity of the encoded portion of the AVR3a homolog. Polynucleotides that are fragments of an AVR3a homolog nucleotide sequence comprise at least 16, 20, 50, 75, 100, 125, 150, 175, 200, 250, 300, 325, 350, 375, 400, or 420 contiguous nucleotides, or up to the number of nucleotides present in a full-length an AVR3a homolog disclosed herein.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the modified R proteins or AVR3a homologs of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a modified R protein or an AVR3a homolog of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptide of SEQ ID NO: 4, 6, 8, 10, 12, 14, 16, 18, 20, 22, 24, 26, 28, 30, 32, 34, 36, 43, 45, 47, 49, 51, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Preferred, variant proteins encompassed by the present invention are biologically active, that is they continue to possess the desired biological activity of the native protein. For the modified R protein, the preferred biological activity is HR activity in a plant, plant part, plant cell in the presence of AVR3a^{KI} and optionally also comprise HR activity in a plant, plant part, plant cell in the presence of AVR3a^{EM} as described herein. For the AVR3a homologs, the preferred biological activity is the capability of inducing HR activity in a plant, plant part, plant cell in the presence of at least one R protein as described herein. Such variants may result from, for example, genetic polymorphism or from human manipulation. Biologically active variants of a modified R protein or AVR3a homolog of the invention will have at least about 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

Preferably, the fragments and variants of a modified R3a protein and other modified R proteins of the present invention will possess 1, 2, 3, 4, 5, 6, 7, or more of the amino acids substitutions (relative to the wild-type R3a protein) of the modified R3a protein and comprise HR activity in a plant, plant part, or plant cell in the presence of AVR3a^{EM}. More preferably, the fragments and variants of a modified R3a protein and other modified R protein of the present invention will possess at least one amino acid substitution that is in the leucine-rich repeat (LRR) domain of the modified R3a protein and comprise HR activity in a plant, plant part, or plant cell in the presence of AVR3a^{EM}. Most preferably, the fragments and variants of a modified R3a protein and other modified R protein of the present invention will possess at least one amino acid substitution that is in the leucine-rich repeat (LRR) domain of the modified R3a protein and comprise HR activity in a plant, plant part, or plant cell in the presence of AVR3a^{EM}, AVR3a^{KI} or both AVR3a^{EM} and AVR3a^{KI}. The amino acid substitutions (relative to the wild-type R3a) of the modified R3a proteins are summarized in FIG. 3. The present invention also encompasses the polynucleotides that encode such fragments and variants.

In certain embodiments of the invention, the modified R3a proteins of comprise HR activity in a plant, plant part, or plant cell in the presence of AVR3a^{KI} and/or AVR3a^{EM} and at least one AVR3a homolog from a Phytophthora species other than P. infestans. The present invention encompasses fragments and variants of such modified R3a proteins that r comprise HR activity in a plant, plant part, or plant cell in the presence of AVR3a^{KI} and/or AVR3a^{EM} and at least one AVR3a homolog from a Phytophthora species other than P. infestans and further encompasses polynucleotides that encode such fragments and variants.

The proteins of the invention may be altered in various ways including amino acid substitutions, deletions, truncations, and insertions. Methods for such manipulations are generally known in the art. For example, amino acid sequence variants and fragments of the polynucleotide R proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms Likewise, the proteins of the invention encompass both naturally occurring proteins as well as variations and modified forms thereof. Such variants will continue to possess the desired biological activity of the modified R protein, particularly the ability to initiate in a plant a hypersensitive response in the presence of an effector protein from a plant pathogen. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by R protein activity assays. See, for example, Van der Hoorn et al. (2000) Mol. Plant-Microbe Interact. 13:439-446; Bos et al. (2006) Plant J. 48:165-176; Bos et al. (2009) Mol. Plant-Microbe Interact. 22: 269-281; herein incorporated by reference.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotides of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode modified R proteins or AVR3a homologs and which hybridize under stringent conditions to at least one of the modified R polynucleotides or AVR3a homolog polynucleotides disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among the sequence of the gene or cDNA of interest sequences and are optimally at least about 10 nucleotides in length, and most optimally at least about 20 nucleotides in length. Such probes may be used to amplify corresponding polynucleotides for the particular gene of interest from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

It is recognized that the modified R protein polynucleotide molecules and modified R proteins of the invention encompass polynucleotide molecules and proteins comprising a nucleotide or an amino acid sequence that is sufficiently identical to the nucleotide sequence of SEQ ID NOS: 1 and/or 3, or to the amino acid sequence of SEQ ID NO: 2. It is further recognized that the polynucleotide molecules and proteins of the invention encompass polynucleotide molecules and proteins comprising a nucleotide or an amino acid sequence that is sufficiently identical to the nucleotide sequence of SEQ ID NOS: 3, 5, 7, 9, 11, 13, 15, 17, 19, 21, 23, 25, 27, 29, 31, 33, 35, 42, 44, 46, 48, 50, and/or 52 or to the amino acid sequence of SEQ ID NO: 4, 6, 8, 10, 12, 14, 16, 18, 20, 22, 24, 26, 28, 30, 32, 34, 36, 43, 45, 47, 49, 51, and/or 53.

It is further recognized that the AVR3a homolog polynucleotide molecules and AVR3a homolog proteins of the invention encompass polynucleotide molecules and proteins comprising nucleotide sequences that encode an amino acid sequence that is sufficiently identical to the amino sequence of SEQ ID NOS: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, and/or 84 or an amino acid sequence that is sufficiently identical to the nucleotide sequence of SEQ ID NOS: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84.

The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 90%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website: http://www.ebi.ac.uk/Tools/clustalw/index.html).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The modified R polynucleotide or AVR3a homolog of the invention comprising modified R protein or AVR3a homolog coding sequences can be provided in expression cassettes for expression in the plant or other organism or non-human host cell of interest. The cassette will include 5′ and 3′ regulatory sequences operably linked to a modified R or AVR3a homolog polynucleotide of the invention. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the modified R or AVR3a homolog polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a modified R or AVR3a homolog polynucleotide of the invention, and a transcriptional and translational termination region (i.e., termination region) functional in plants or other organism or non-human host cell. The regulatory regions (i.e., promoters, transcriptional regulatory regions, and translational termination regions) and/or the modified R or AVR3a homolog polynucleotide or of the invention may be native/analogous to the host cell or to each other. Alternatively, the regulatory regions and/or the modified R polynucleotide or AVR3a homolog polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the modified R coding sequences using heterologous promoters, the native promoter sequence of the unmodified R gene may be used.

The termination region may be native with the transcriptional initiation region, may be native with the operably linked modified R polynucleotide or AVR3a homolog polynucleotide of interest, may be native with the plant host, or may be derived from another source (i.e., foreign or heterologous) to the promoter, the modified R polynucleotide or AVR3a homolog polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the modified R polynucleotide or AVR3a homolog polynucleotide within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant. Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Bairn et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

The methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed. In an embodiment of the invention, modified R polynucleotide is operably linked to a plant promoter that is known for high-level expression in a plant cell, and this construct is then introduced into a plant that is susceptible to an imidazolinone herbicide and a transformed plant is regenerated. The transformed plant is tolerant to exposure to a level of an imidazolinone herbicide that would kill or significantly injure an untransformed plant. This method can be applied to any plant species; however, it is most beneficial when applied to crop plants.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. It is recognized that the a modified R protein of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In specific embodiments, the modified R sequences or AVR3a homolog sequences of the invention can be provided to a plant using a variety of transient transformation methods. Such transient transformation methods include, but are not limited to, the introduction of the modified R protein or variants and fragments thereof, or AVR3a homolog proteins variants and fragments thereof, directly into the plant or the introduction of a modified R or AVR3a homolog transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described below.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

The present invention may be used for transformation of any plant species, including, but not limited to, monocots and dicots. Examples of plant species of interest include, but are not limited to, peppers (Capsicum spp; e.g., Capsicum annuum, C. baccatum, C. chinense, C. frutescens, C. pubescens, and the like), tomatoes (Lycopersicon esculentum), tobacco (Nicotiana tabacum), eggplant (Solanum melongena), petunia (Petunia spp., e.g., Petunia×hybrida or Petunia hybrida), corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), palms, oats, barley, vegetables, ornamentals, and conifers.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruits, roots, root tips, anthers, and the like. Progeny, variants, and mutants of the regenerated plants are also included within the scope of the invention, provided that these parts comprise the introduced polynucleotides. As used herein, “progeny” and “progeny plant” comprise any subsequent generation of a plant unless it is expressly stated otherwise or is apparent from the context of usage.

The invention is drawn to compositions and methods for enhancing the resistance of a plant to plant disease. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen is minimized or lessened.

The following examples are offered by way of illustration and not by way of limitation.

### EXAMPLES

### Example 1

### Modification of the R3a Resistance Protein to Extend Recognition to Effector Proteins Encoded by Virulent Alleles of Avr3a from Phytophthora infestans

Phytophthora infestans is one of the most devastating pathogens affecting potato production worldwide. One strategy to generate resistant cultivars is the introduction of resistance genes that are able to recognize P. infestans effector proteins with avirulence activities. R3a, a resistance protein discovered in potato, can trigger an hypersentive response upon the recognition of the avirulence effector AVR3a^{KI} from P. infestans but cannot recognize AVR3a^{EM}, the product of another allele that is predominant in pathogen populations. To date, all the characterized P. infestans strains in nature carry at least one of these AVR3a proteins.

### Materials and Methods

### Obtaining R3a Mutant Versions

The S. tuberosum R3a resistance gene (Huang et al. (2005) Plant J. 42: 251-261) was used as the template for a PCR-based random mutagenesis protocol (Diversify PCR Random Mutagenesis Kit, Clontech, Takara Bio Company) following the supplier's protocol. Primers were designed to amplify R3a including restriction enzymes recognition sites for the subcloning step (R3a_BamHI_Fw_MES: GGAGGATCCATGGAGATTGGCTTAGCAG, SEQ ID NO: 38 and R3a_SpeI_Rev_MES: GGAACTAGTTCACATGCATTCCCTATC, SEQ ID NO: 39). Different conditions were tested to generate various mutation rates. We selected those conditions that gave us a mutation rate ranging: between 1 and 2 mutations every 1000 bp (3-8 in a 4000 bp gene). The PCR reaction was purified using a QIAquick PCR Purification Kit (QIAGEN) and kept for later cloning steps.

### Generation of the Mutagenized R3a Library

PCR purified product (R3a mutagenized molecules; coding sequence only) and pCB302-3 binary vector (Xiang et al. (1999) Plant Mol. Biol. 40: 711-717) were digested with BamH I and Spe I restriction enzymes (Roche Applied Science). Digested PCR products and vector were gel-purified using the kit Wizard® SV Gel and PCR Clean-Up System (Promega), ligated and ligation mixture was transformed into electro competent A. tumefaciens GV3101 cells to make a library “ready-to-use” in screening assays with the R3a mutagenized molecules under the control of the CaMV 35S promoter. The transformation efficiency was >5500 ufcs/ml, with more than 75% of positive clones. The colonies were picked out using a QPix colony-picking robot (Genetix, New Milton, U.K.) into 384 wells plates for preparation of freezer stocks. The library, containing more than 6000 clones (19 plates of 384 wells each), was kept at −80° C.

### Screening of the Library

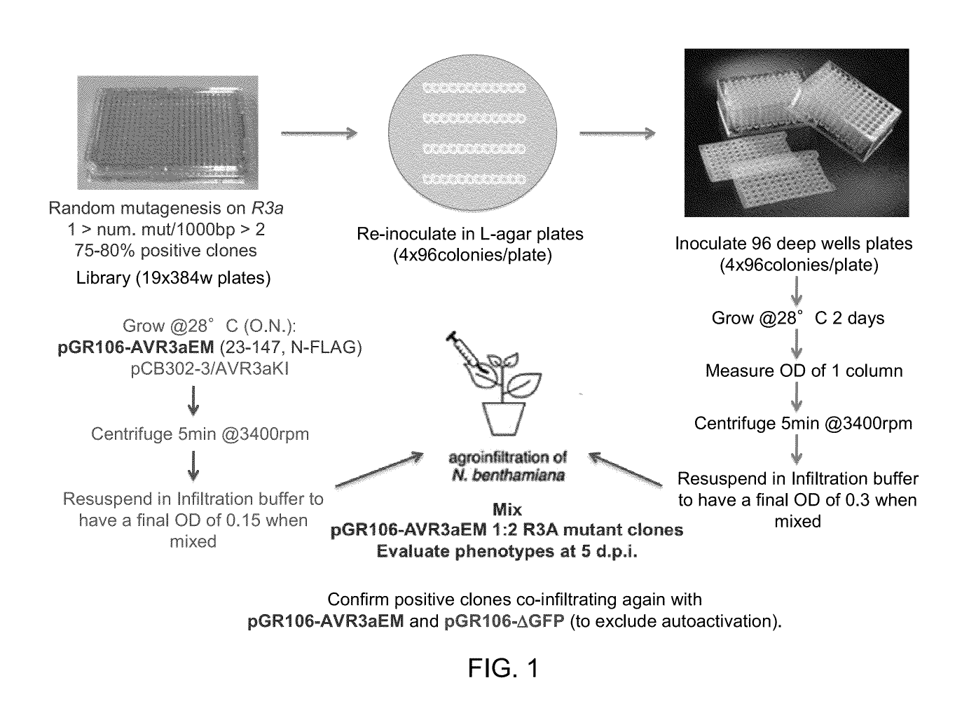

The screening was performed using the agroinfiltration transient assay in Nicotiana benthamiana (Van der Hoorn et al. (2000) Mol. Plant-Microbe Interact. 13:439-446; Bos et al. (2006) Plant J. 48:165-176; Bos et al. (2009) Mol. Plant-Microbe Interact. 22: 269-281). 96 wells 2 ml-deph plates containing 500 μl of LB media with antibiotics (rifampicin 50 mg/L, gentamicin 20 mg/L and kanamicin 50 mg/L) were inoculated with the library clones and grew at low speed and 28° C. for 48 hrs (to reach an OD600 of 1-1,2). Cultures were pelleted by centrifugation (5 min at 3500 rpm and 15° C.) and resuspended with infiltration buffer (1 L MMA: 5 g MS salts, 1.95 g MES, 20 g sucrose, 200 μM acetosyringone, pH 5.6) to a final OD600 of 0.6. A. tumefaciens GV3101 transformed with pGR106-AVR3a_K80I103 and pRG106-AVR3a_E80M103 (Armstrong et al. (2005) PNAS 102:7766-7771) were grown for agroinfiltration as previously described (Van der Hoorn et al. (2000) Mol. Plant-Microbe Interact. 13:439-446), except that the culturing steps were performed in LB media supplemented with rifampicin 50 mg/L, gentamicin 20 mg/L and kanamicin 50 mg/L. Cultures were pelleted and resuspended in infiltration buffer as described above. For transient co-expression of R3a, R3a mutated clones and AVR3a, the cells resuspended in infiltration buffer were mixed to have a final OD600=0.3 or 0.15 for R3a clones and AVR3a clones respectively. A. tumefaciens GV3101 transformed with pGR106-ΔGFP (it contains a truncated version of gfp) was grew as the AVR3a clones. Agroinfiltration experiments were performed on 4 to 6-week-old N. benthamiana plants. Plants were grown and maintained throughout the experiments in a controlled environment room with an ambient temperature of 22 to 25° C. and high light intensity. Symptom development was monitored from 3 to 8 days after infiltration (d.p.i.). Each R3a mutant clone was co-infiltrated with Avr3a^{EM} for gain of function assessment. Interesting clones were co-infiltrated with Avr3a^{KI} for loss of function assessment. After the first round of screening, the clones that gave a positive response with AVR3a^{EM} were selected for a new round of agroinfiltration to confirm the previous observations and to rule out auto activation. The strategy that was used is summarized in FIG. 1.

### Validation of the Candidate Clones

The positive clones selected after the first round of infiltrations were infiltrated again with Avr3a^{EM} or a control vector (pGR106-ΔGFP) to rule out auto activation. After the first round of screening (2200 clones) and validation, 19 clones that showed a clear response to AVR3a^{EM} but not with the control vector were selected for further analysis.

### R3a Mutant Clones Characterization: Comparative Effector Recognition

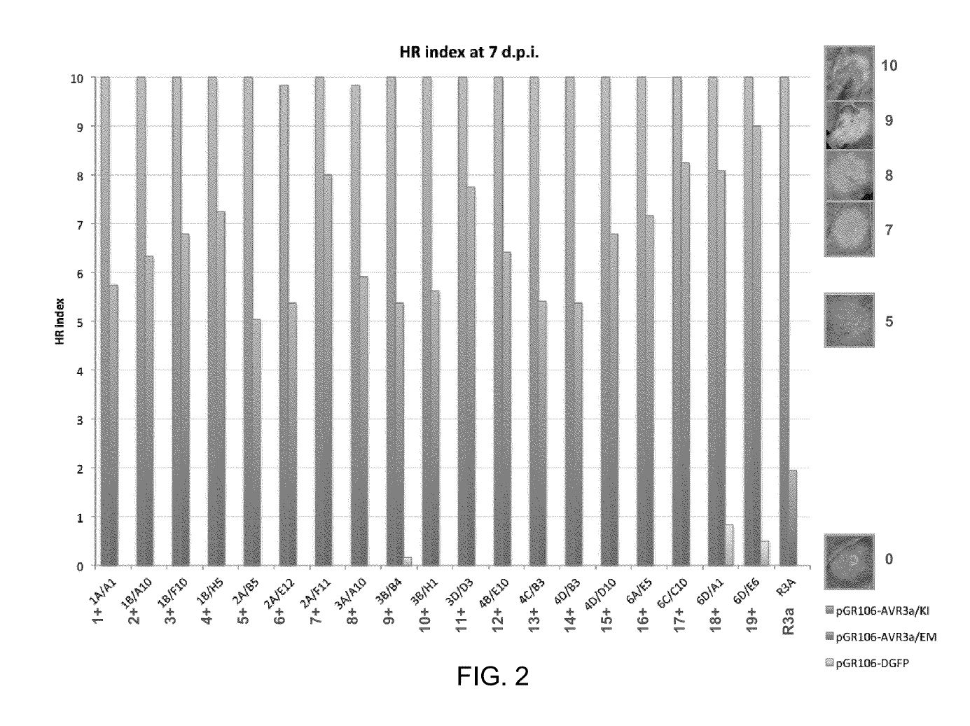

The selected clones were co-infiltrated in N. benthamiana leaves to compare their relative response when AVR3aEM, AVR3aKI or AGFP are present. Co infiltrations were performed as mentioned above. Each combination of R3a mutant-clone and AVR3a (or Δgfp) was infiltrated as 10 to 12 replicates each. HR-like phenotype was scored in a daily basis up to 8 d.p.i., according to an arbitrary scale from 0 (no phenotype observed) to 10 (confluent necrosis). Results are summarized in FIG. 2.

### R3a Mutant Clone Characterization—Sequence Analysis of the Candidate Clones

Plasmid DNA from 17 of the candidate clones was isolated and R3a inserts were sequenced using several primers to allow full coverage. The analysis of the sequences allowed the identification of several mutations in each clone.

### R3a Mutant Clones Characterization—Differential Contribution of the Mutations to the Observed HR-Like Phenotype

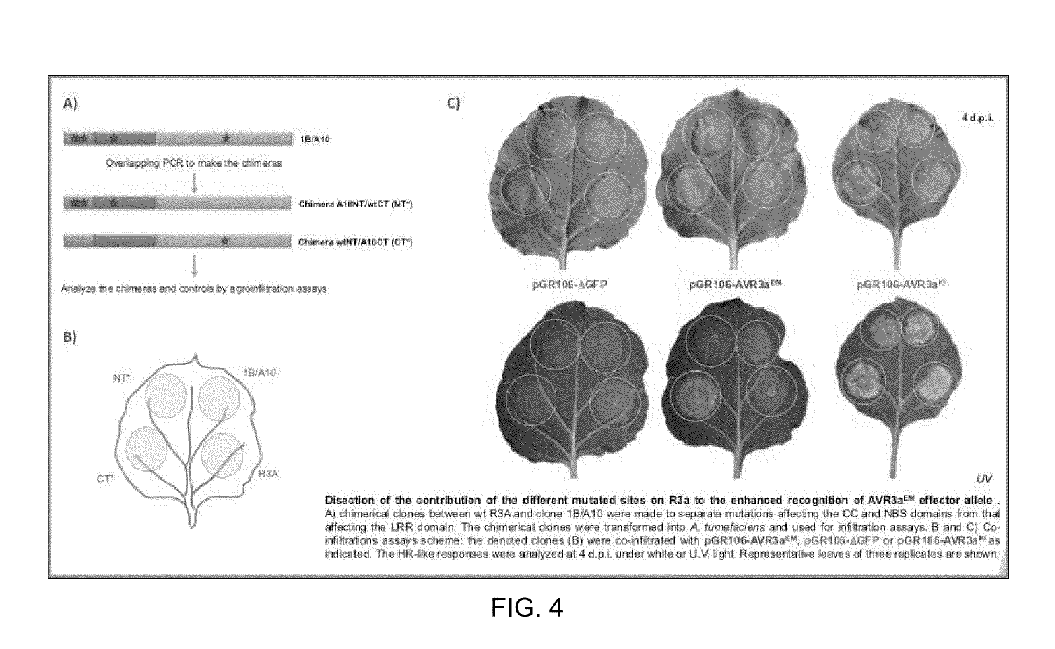

To assess if particular mutations out of all the mutations present in each clone are responsible for the extended recognition specificity, we made chimerical clones between R3a wt and clone 1B/A10 by overlapping PCR (FIG. 4). After amplification, PCR products were digested with BamH I and Spe I restriction enzymes, gel purified and ligated into pCB302-3 binary vector as already described but with the chimerical clones operably linked to a the constitutive Rip.vnt1.1 promoter (SEQ ID NO: 40; Foster et al. (2009) MPMI 22:589-600) and the Rip.vnt1.1 terminator (SEQ ID NO: 41; Foster et al. (2009) MPMI 22:589-600). The result were two clones, named NT* and CT*. NT* contains the four amino acid substitutions in the CC and NBS domains of 1B/A10, while CT* contains only one amino acid substitution in the LRR domain. The nucleotide and amino acid sequences of CT* are set forth in SEQ ID NOS: 52 and 53, respectively. These two clones were transformed into A. tumefaciens GV3101 electrocompetent cells for agroinfiltration experiments. Clones 1B/A10, NT*, CT* and R3a wt (all having pCB302-3 as the backbone vector) were co-infiltrated with pGR106-Avr3aKI, pGR106-Avr3aEM or pGR106-ΔGFP using the same methodology already explained.

### Results

To attempt to extend R3a recognition specificity to AVR3a^{EM}, a library of R3a mutant variants was produced by random mutagenesis. The mutated nucleic acid molecules were cloned in a T-DNA binary vector and transformed into Agrobacterium tumefaciens. The mutant clones were screened by co-agroinfiltration with AVR3a^{EM} in Nicotiana benthamiana plants, and evaluated the presence of HR-like phenotypes after 5 days.

Of approximately 2200 evaluated clones, 20 triggered different degrees of HR-like responses and were subjected to new rounds of infiltrations to confirm the results. In parallel, the candidate clones were co-infiltrated with AVR3a^{KI} and with a negative control plasmid to investigate the conservation of the original R3a recognition specificity and also to eliminate auto-active R3a mutants. In total, 17 clones were selected for further analyses, including sequencing and the construction of chimerical clones to investigate which mutations are responsible for the observed phenotypes.

As observed in FIG. 2, the 19 clones showing a response to AVR3a^{EM} show a different degree of HR-like phenotype, but in all the cases, it was higher than the response observed with the wild-type R3a resistance protein. Moreover, all the clones showed recognition specificity for AVR3a^{KI}, and in a few cases, a minor response was observed against the Δgfp construct. In the analyzed cases, mutations selected extended the recognition specificity of the mutated R3a clones towards AVR3a^{EM} without affecting the original recognition of AVR3a^{KI}, and without triggering auto-activation of R3a.

Sequences from the mutant R3a clones capable of recognizing AVR3a^{EM} were obtained and mutations compared to wild type R3a identified. Many of the mutations were at positions giving rise to amino acid changes. The relative position of the mutations in each clone and the mutations itself are summarized in FIG. 3.

As observed in FIG. 4, only the mutation in the LRR domain is the one that confers to clone 1B/A10 the new recognition specificity towards AVR3a^{EM}. Moreover, when only this amino acid change is present (E941K), the HR response is stronger than the one observed with the original clone. Interesting mutations seem to be located in the LRR domain, as observed with the chimerical clone CT* and also with clone 6C/C10 (K920E). The nucleotide and amino acid sequences of 6C/C10 are set forth in SEQ ID NOS: 31 and 32, respectively. These results indicate that individual amino acid changes can confer R3a an extended recognition specificity, and also that mutations with negative impact on AVR3aEM recognition could be depurated from the candidate clones, as shown for clone 1B/A10.

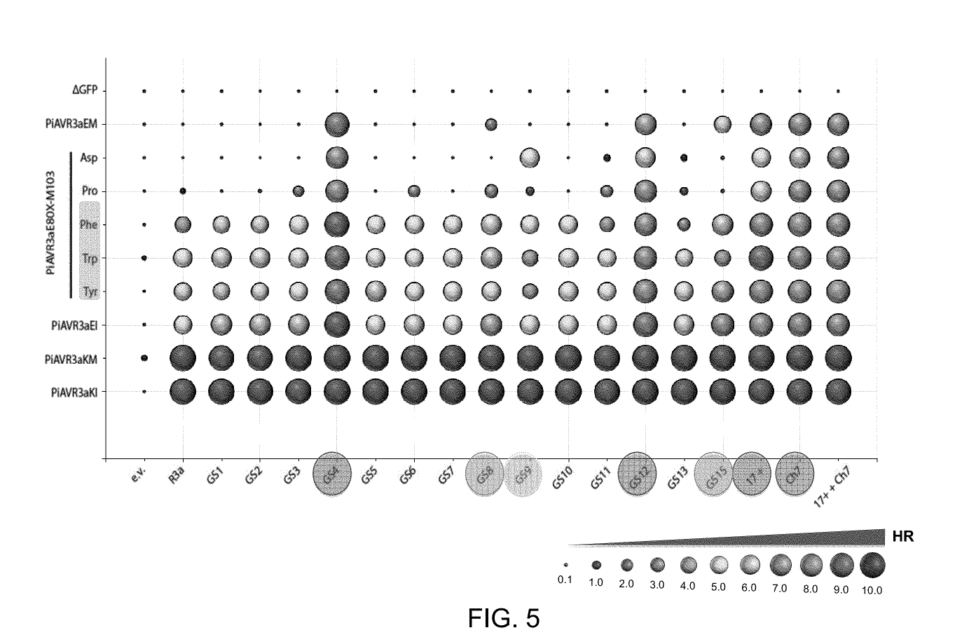

In addition, several other single amino acid substitutions in the LRR domain of R3a have been shown to confer recognition specificity towards AVR3a^{EM} on R3a. (FIG. 5). These amino acid substitutions include, for example, L668P, C950R, E983K, and K1250R (Table 1).

TABLE 1
Additional Single Amino Acid Substitutions
that Confer Recognition of AVR3a^{EM} on R3a

| | Amino acid | Amino Acid | Nucleotide |
|---|---|---|---|
| Clone ID | change | Sequence | Sequence |
| GS4 | L668P | SEQ ID NO: 43 | SEQ ID NO: 42 |
| GS8 | C950R | SEQ ID NO: 45 | SEQ ID NO: 44 |
| GS12 | E983K | SEQ ID NO: 49 | SEQ ID NO: 48 |
| GS15 | K1250R | SEQ ID NO: 51 | SEQ ID NO: 50 |

### Example 2

### Modified R3a Proteins Recognized AVR3a Homologs in Other Phytophthora Species

AVR3a is polymorphic and homologs are present in at least three Phytophthora species, P. infestans, P. capsici and P. sojae. AVR3a homologs from P. capsici and P. sojae were cloned, and their ability to trigger R3a-mediated HR and suppression of INF-1 induced cell death was assessed (Bos (2007) “Function and evolution of the RxLR effector AVR3a of Phytophthora infestans”, Ph.D. Dissertation, The Ohio State University). Most homologs did not display AVR3a-like effector activity, except the homologs from P. infestans PEX147-3 (PiPEX147-3) and P. sojae AVH1b (PsAVH1b), both of which were able to induce a HR upon co-expression in N. benthamiana.

Co-infiltration experiments in N. benthamiana were conducted essentially as described in Example 1 to investigate if modified R3a+ proteins of the present invention could recognize not only AVR3a^{EM} from P. infestans but also one or more AVR3a homologs from other Phytophthora species (FIGS. 6-7). The results of these experiments revealed that 6D/A1 and 6D/E6 recognize P. capsici AVR3a11 (PcAVR3a11) and 6D/A1, 6D/E6, and 4D/B3 showed enhanced recognition towards AVR1b from P. sojae (PsAVR1b) (FIG. 6). Moreover, CT* with a single amino acid substitution (E941K) showed the new recognition specificity for PsAVR1b (FIG. 7). All of the tested clones recognized PiPEX147-3 and PsAVH1b in a similar way as the wild-type R3a protein. For PcAVR3a4, most of the clones behaved like the wild-type R3a protein except for 2A/B5 and 6C/C10 (FIG. 6). The hypersentive response triggered by PcAVR3a4 when co-infiltrated with these clones was reduced when compared to the hypersentive response with the wild-type R3a protein.

In summary, some modified R3a proteins of the present invention have expanded recognition specificity and can also recognize AVR3a homologs from other Phytophthora species.

### Example 3

### Modified R3a Protein GS4 Triggers a Stronger Hypersensitive Response in the Presence of PiAVR3a^{KI} than Wild-Type R3a

Several R3a modified clones (GS4, 8, 12 and 15; 6C/C10 and Ch7) or the wt R3a clone (all cloned in the pCBNptII_PTvnt1.1 backbone) were co-infiltrated side-by-side with serial dilutions of PiAVR3aKI (pK7 backbone) in N. benthamiana leaves essentially as described in Example 1. An empty vector (EV) clone was included as a control. The phenotype (HR) was scored in one of three categories at 4 d.p.i. for neighboring spots one the same leaf (i.e., modified R3a and wt R3a in opposite sides of the leaf) as follows: (1) modified R3a stronger than R3a, (2) modified R3a equal to R3a, or (3) modified R3a weaker than R3a.

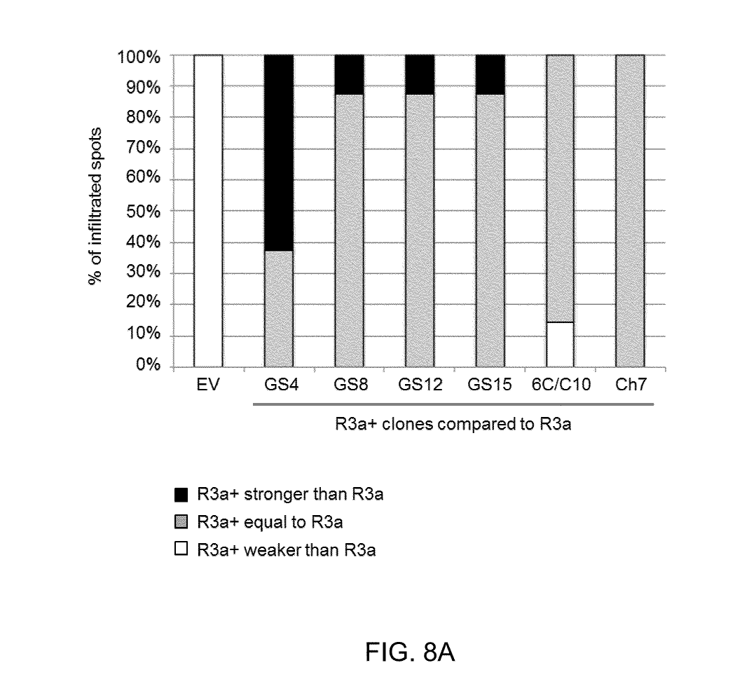

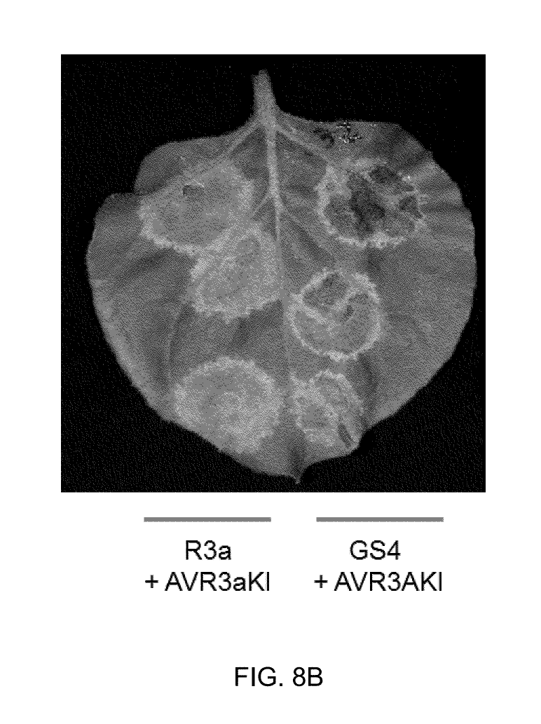

The results for the lowest concentration of PiAVR3aKI are plotted as percentage of compared spots showing each of the possible outcomes in FIG. 8A. GS4 gave a stronger HR for approximately 60% of the infiltrated spots, suggesting that the modified R3a protein encoded by this clone not only has expanded recognition specificity towards PiAVR3a^{EM} and AVR3a family members for other Phythophthora species but is also more sensitive for PiAVR3a^{KI} allele recognition, when compared to the wild-type R3a protein. A representative picture is included (FIG. 8B).

### Example 4

### Co-Expression of Modified R3a Proteins with Wild-Type R3a Protein

The GS4 and GS12 clones, each of which encodes a modified R3a protein, were separately co-infiltrated into N. benthamiana leaves with a clone encoding R3a (wild-type) and a clone encoding AVR3a^{EM}. The co-infiltrations were conducted essentially as described in Example 1, and HR was evaluated at 2.5, 3.5, 4.5, and 5.5 d.p.i.

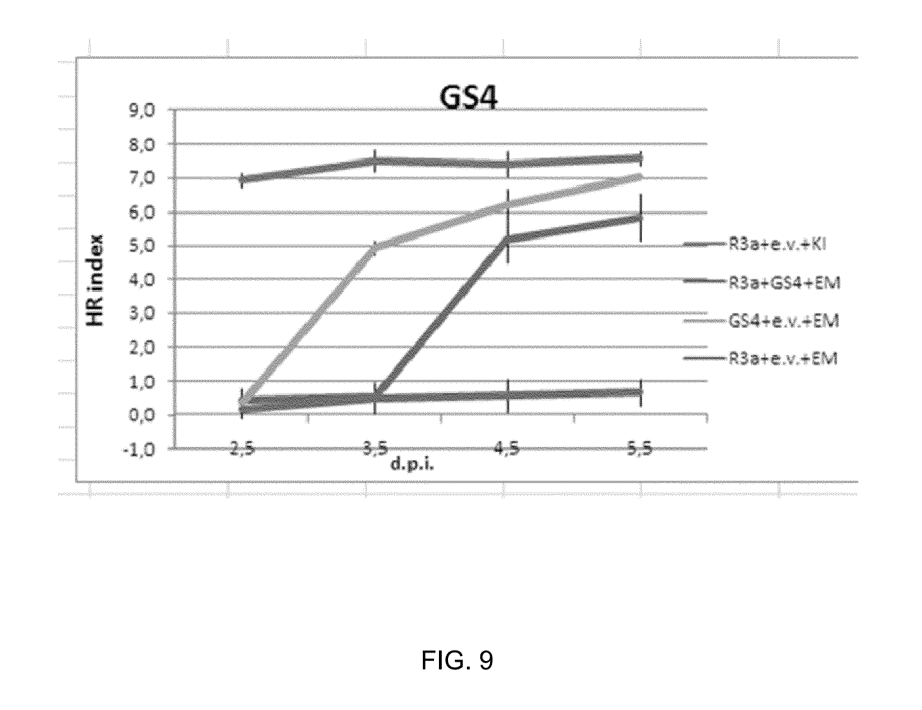

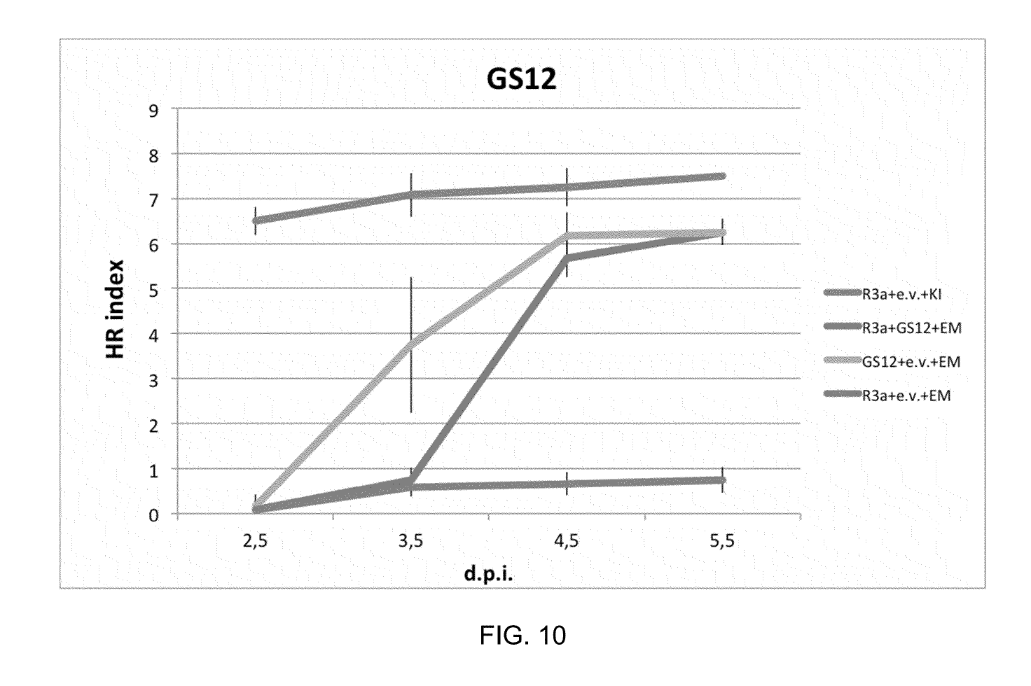

The results for GS4 and G12 are shown in FIGS. 9 and 10, respectively. Relative to the co-infiltration of GS4, empty vector (e.v.), and PiAVR3a^{EM}, HR was delayed when R3a, GS4, and PiAVR3a^{EM} were co-infiltrated together (FIG. 9). Similar results were obtained with GS12 (FIG. 10). While the present invention does not depend on a particular biological mechanism, it is recognized that the results shown in FIGS. 9 and 10 suggest that the R3a protein may act in vivo as a dimer.

### Example 5

### A Modified R3a Protein Triggers a Hypersensitive Response in the Presence of AVR3a Homologs from Phytophthora palmivora in Leaves from Both Solanaceous and Non-Solanaceous Plants

Biotrophic pathogens specialize on a few related host plants. However, Phytophthora palmivora, a ubiquitous tropical fungal-like oomycete can infect more than 200 host species and is a threat for chocolate producing countries because it causes pod rot on cocoa (Theobroma cacao). Characterised plant disease resistance proteins only confer resistance to specific pathogens by targeted recognition of single effector proteins. However, Phytophthora effectors evolved to overcome plant perception.

To obtain target effectors for known resistance genes an aggressive strain of Phytophthora palmivora from Colombia was sequenced and used for assembly and blast analysis against known avirulence genes. Five different candidates with homology to AVR3a of P. infestans were identified. Degenerate primers were used to amplify a full set of paralogs from the genome and 15 different AVR3a variants harbouring 9 different effector domains were identified.

To test whether the previously characterised R3a protein could be adapted to recognise a wider spectrum of AVR3a-related effectors, including Phytophthora palmivora AVR3a variants, an R3a mutant library was screened for variants with extended specificity and the modified R3a protein of clone GS4 was identified. Nine different Phytophthora palmivora AVR3a effector domains were tested with the GS4 R3a protein in co-infiltration assays in N. benthamiana essentially as described in Example 1. Seven different Phytophthora palmivora AVR3a homologs were found to trigger a hypersensitive response with GS4 R3a protein (Table 2). Generally, the GS4 R3a protein seems to enhance timing and intensity of HR development. Furthermore, it was determined that the GS4 R3a protein confers recognition of variants L3B and L3C which were unrecognized by the native R3a protein confirming GS4 R3a protein's increased potential for AVR3a effector family recognition.

TABLE 2
Cell Death/HR Intensity Upon Coexpression of P. palmivora
AVR3a Homologs with Different R3a Variants,

| | | pVnt1-R3a | | | pBIN::35S-R3a | | | pVnt1-R3aGS4 | | | |
|---|---|---|---|---|---|---|---|---|---|---|---|
| Variant | ev | 1dpi | 2dpi | 3dpi | 1dpi | 2dpi | 3dpi | 1dpi | 2dpi | 3dpi | |
| L2A | 0 | 2 | 3 | 4 | 2 | 2 | 3 | 2 | 4 | 4 | |
| L3B | 0 | 0 | 0 | 0 | 0 | 1 | 2 | 0 | 2 | 3 | * |
| L3C | 0 | 0 | 0 | 0 | 0 | 0 | 0 | 2 | 3 | 3 | ** |
| L4A | 0 | 0 | 1 | 2 | 0 | 1 | 2 | 0 | 1 | 2 | |
| L5A | 0 | 0 | 1 | 3 | 0 | 1 | 3 | 2 | 3 | 4 | |
| L6B | 0 | 0 | 0 | 0 | 0 | 0 | 0 | 0 | 0 | 0 | |
| L7A | 0 | 0 | 0 | 1 | 0 | 0 | 1 | 0 | 0 | 2 | |
| L7B | 0 | 0 | 1 | 1 | 0 | 1 | 1 | 2 | 2 | 2 | |
| L7C | 0 | 0 | 0 | 0 | 0 | 0 | 0 | 0 | 0 | 0 | |
| AVR3a^{KI} | 0 | 2 | 3 | 4 | 1 | 1 | 2 | 2 | 4 | 4 | |

Asterisks indicate significant gain in recognition only upon use of the GS4 R3a protein.

Tests were conducted to determine whether the GS4 R3a protein can limit P. palmivora infection in N. benthamiana. The GS4 R3a protein was found to effectively block infection of two different P. palmivora isolates suggesting that both isolates carry AVR3a homologs (Table 3).

TABLE 3
Infection Assays of P. palmivora and P. infestans
on N. benthamiana Transgenic for Expression of the
Wild-Type R3a Protein or the GS4 Modified R3a Protein

| | | | P. infestans |
|---|---|---|---|
| Construct | Agro AVR3aKI | P. palmivora | T30/4 (TD) |
| Pvnt-R3aGS4 #1 | all strong HR | sign. Reduced | not infected |
| Pvnt-R3aGS4 #4 | all strong HR | sign. Reduced | not infected |
| Vector #4 | all no HR | all infected | all infected |
| Vector #10 | all no HR | all infected | all infected |
| 35S-R3a WT | strong HR | reduced/not inf. | (Not tested) |

AVR3a homologs were amplified from the second isolate and identified 12 different variants. The amino acid sequences of the AVR3a homologs from the two different isolates of Phytophthora palmivora (L=16830, AJ=6390) are set forth in SEQ ID NOS: 54-84.

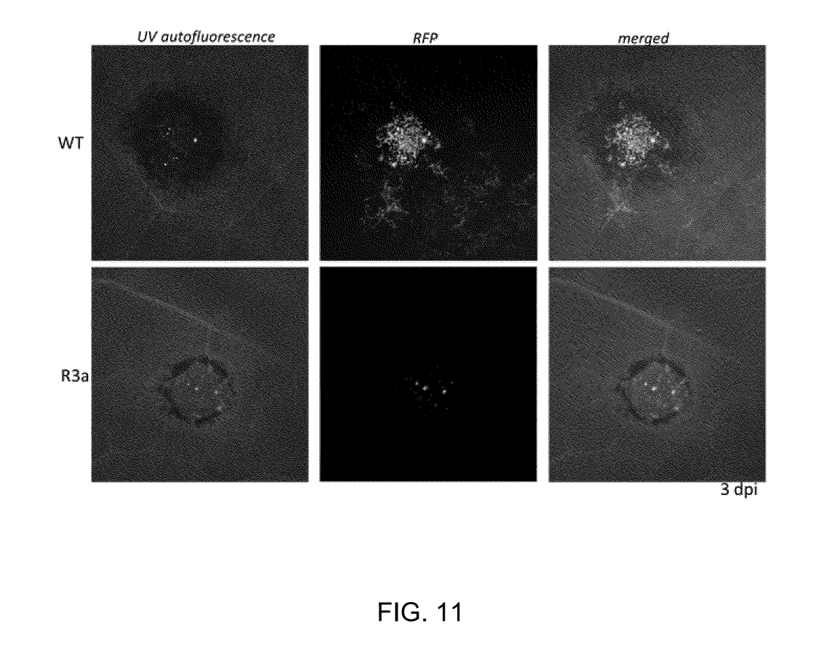

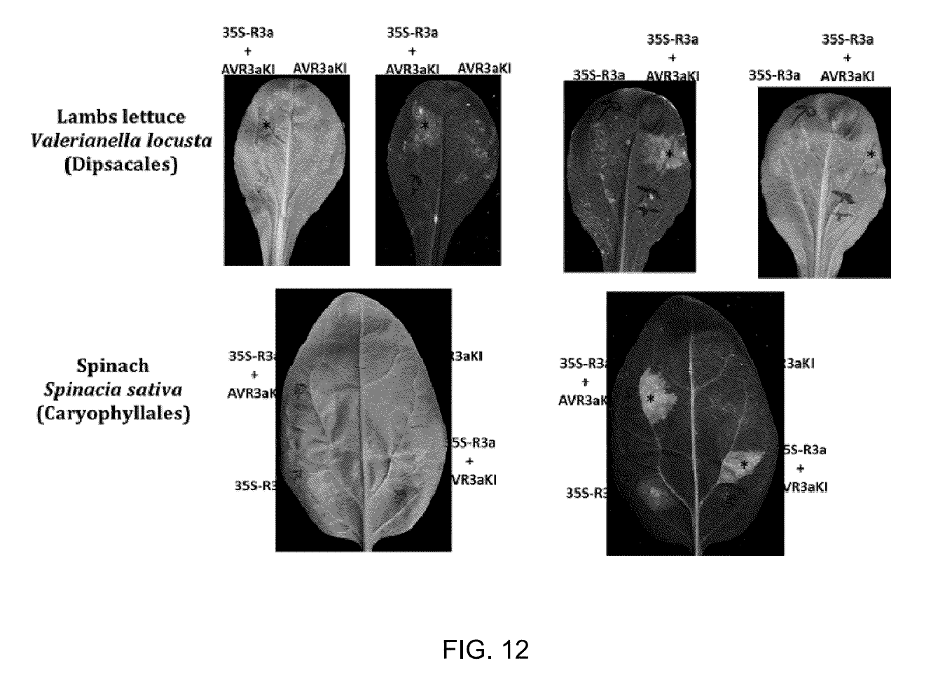

To transfer R3a to other hosts it is crucial that its function is not limited to potato or related Solanaceous plants. Agrobacterium transient expression was used to test its ability to mediate AVR3a recognition in taxonomically unrelated species essentially as described in Example 1. It was determined that HR induction is maintained in lamb's lettuce (Valerianella locusta) and spinach (Spinacia oleracea), suggesting applicability of R3a in unrelated non-Solanaceous host plants as shown in FIGS. 11-12.

The results described in this example provide a framework for genome-aided identification of Phytophthora effector proteins and development of extended specificity disease resistance proteins. A variant of the R3a disease resistance protein (GS4) was produced and determined to have the ability to confer resistance towards isolates of P. palmivora thru recognition of an extended set of multiple AVR3a-like effectors.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A nucleic acid molecule comprising a nucleotide sequence selected from the group consisting of:
(a) a nucleotide sequence encoding a modified R3a protein that is capable of inducing a hypersensitive response in a plant in the presence of AVR3a^{EM};
(b) a nucleotide sequence encoding a modified R3a protein that is capable of inducing a hypersensitive response in a plant in the presence of AVR3a^{EM} and that is capable of inducing a hypersensitive response in a plant in the presence of AVR3a^{KI};
(c) the nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52;
(d) a nucleotide sequence encoding the amino acid sequence set forth in SEQ ID NO: 4, 6, 8, 10, 12, 14, 16, 18, 20, 22, 24, 26, 28, 30, 32, 34, 36, 43, 45, 47, 49, 51, or 53;
(e) a nucleotide sequence comprising at least 85% nucleotide sequence identity to at least one nucleotide sequence selected from the group consisting of SEQ ID NOS: 3, 5, 7, 9, 11, 13, 15, 17, 19, 21, 23, 25, 27, 29, 31, 33, 35, 42, 44, 46, 48, 50, and 52, wherein said nucleotide molecule encodes a protein comprising HR activity in a plant in the presence of AVR3a^{EM};
(f) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM};
(g) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions as set forth in FIG. 3;
(h) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions in the LRR domain as set forth in FIG. 3;
(i) a fragment of the nucleotide sequence of any one of (a)-(h), wherein said fragment encodes a protein comprising HR activity in a plant in the presence of AVR3a^{EM};
(j) the nucleotide sequence of any one of (e)-(i), wherein said protein further comprises HR activity in a plant in the presence of AVR3a^{KI}; and
(k) a nucleotide sequence that is fully complementary to the nucleotide sequence of any one of (a)-(j).

2. An expression cassette comprising the nucleic acid molecule of claim 1 operably linked to a promoter.

3. A non-human host cell comprising the expression cassette of claim 2.

4. A plant or plant cell comprising the expression cassette of claim 2.

5. A plant comprising in its genome a heterologous polynucleotide, said heterologous polynucleotide comprising a nucleotide sequence encoding a modified R3a protein and an operably linked to promoter capable of driving expression of said nucleotide sequence in a plant, wherein said modified R3a protein is capable of inducing a hypersensitive response in a plant in the presence of AVR3a^{EM} and optionally is capable of inducing a hypersensitive response in a plant in the presence of AVR3a^{KI}.

6. The plant of claim 5, wherein said nucleotide sequence comprises a nucleotide sequence selected from the group consisting of:
(a) the nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52;
(b) a nucleotide sequence encoding the amino acid sequence set forth in SEQ ID NO: 4, 6, 8, 10, 12, 14, 16, 18, 20, 22, 24, 26, 28, 30, 32, 34, 36, 43, 45, 47, 49, 51, or 53;
(c) a nucleotide sequence comprising at least 85% nucleotide sequence identity to at least one nucleotide sequence selected from the group consisting of SEQ ID NOS: 3, 5, 7, 9, 11, 13, 15, 17, 19, 21, 23, 25, 27, 29, 31, 33, 35, 42, 44, 46, 48, 50, and 53, wherein said nucleotide molecule encodes a protein comprising HR activity in a plant in the presence of AVR3a^{EM};
(d) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM};
(e) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions as set forth in FIG. 3;
(f) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions in the LRR domain as set forth in FIG. 3; and
(g) a fragment of the nucleotide sequence of any one of (a)-(f), wherein said fragment encodes a protein comprising HR activity in a plant in the presence of AVR3a^{EM}.

7. The plant of claim 5, wherein the promoter is selected from the group consisting of a constitutive promoters, wound-inducible promoters, pathogen-inducible promoters, chemical-regulated promoters, chemical-inducible promoters, and tissue-preferred promoters.

8. The plant of claim 5, wherein the plant is selected from the group consisting of potato, tomato, eggplant, petunia, Physalis sp., woody nightshade, garden huckleberry, gboma eggplant, Ageratum conyzoides, Solanecio biafrae, peppers, soybean, cocoa, and palms.

9. The plant of claim 5, wherein said plant is a seed.

10. A plant part of the plant of claim 5.

11. The plant part of claim 10, wherein said part is a tuber, a seed, or a fruit.

12. A method for enhancing the resistance of a plant to at least one Phytophthora species, said method comprising transforming a plant cell with a polynucleotide comprising a nucleotide sequence encoding a modified R3a protein, wherein said modified R3a protein is capable of inducing a hypersensitive response in a plant in the presence of AVR3a^{EM} and optionally is capable of inducing a hypersensitive response in a plant in the presence of AVR3a^{KI}.

13. The method of claim 12, wherein the Phytophthora species is selected from the group consisting of P. infestans, P. sojae, P. capsici, and P. palmivora.

14. The method of claim 12, wherein said nucleotide sequence comprises a nucleotide sequence selected from the group consisting of:
(a) the nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52;
(b) a nucleotide sequence encoding the amino acid sequence set forth in SEQ ID NO: 4, 6, 8, 10, 12, 14, 16, 18, 20, 22, 24, 26, 28, 30, 32, 34, 36, 43, 45, 47, 49, 51, or 53;
(c) a nucleotide sequence comprising at least 85% nucleotide sequence identity to at least one nucleotide sequence selected from the group consisting of SEQ ID NOS: 3, 5, 7, 9, 11, 13, 15, 17, 19, 21, 23, 25, 27, 29, 31, 33, 35, 42, 44, 46, 48, 50, and 52, wherein said nucleotide molecule encodes a protein comprising HR activity in a plant in the presence of AVR3a^{EM};
(d) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM};
(e) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions as set forth in FIG. 3;
(f) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions in the LRR domain as set forth in FIG. 3; and
(g) a fragment of the nucleotide sequence of any one of (a)-(f), wherein said fragment encodes a protein comprising HR activity in a plant in the presence of AVR3a^{EM}.

15. The method of claim 12, wherein the plant comprises enhanced resistance to Phytophthora infestans strains comprising AVR3a^{EM} and optionally comprises enhanced resistance to Phytophthora infestans strains comprising AVR3a^{KI}.

16. The method of claim 12, wherein the plant is selected from the group consisting of potato, tomato, eggplant, petunia, Physalis sp., woody nightshade, garden huckleberry, gboma eggplant, Ageratum conyzoides, Solanecio biafrae, peppers, soybean, cocoa, and palms.

17. The method of claim 12, wherein the polynucleotide further comprises an operably linked promoter capable of driving expression of said nucleotide sequence in a plant.

18. The method of claim 12, further comprising regenerating a transformed plant from said transformed cell.

19. A plant or plant cell produced by the method of claim 12.

20. A method for enhancing the resistance of a potato plant to Phytophthora infestans, said method comprising altering the coding sequence of the R3a gene in a plant or plant cell, whereby the altered coding sequence encodes a modified R3a protein that comprises an amino acid sequence having at least one amino acid substitution relative to the amino acid sequence of the R3a protein encoded by the R3a gene, wherein said modified R3a protein is capable of inducing a hypersensitive response in a plant in the presence of AVR3a^{EM} and optionally is capable of inducing a hypersensitive response in a plant in the presence of AVR3a^{KI}.

21. The method of claim 20, wherein altering the coding sequence of the R3a gene in a plant comprises in vivo targeted mutagenesis, homologous recombination, or mutation breeding.

22. The method of claim 20, wherein said altered coding sequence comprises a nucleotide sequence selected from the group consisting of:
(a) the nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52;
(b) a nucleotide sequence encoding the amino acid sequence set forth in SEQ ID NO: 4, 6, 8, 10, 12, 14, 16, 18, 20, 22, 24, 26, 28, 30, 32, 34, 36, 43, 45, 47, 49, 51, or 53;
(c) a nucleotide sequence comprising at least 85% nucleotide sequence identity to at least one nucleotide sequence selected from the group consisting of SEQ ID NOS: 3, 5, 7, 9, 11, 13, 15, 17, 19, 21, 23, 25, 27, 29, 31, 33, 35, 42, 44, 46, 48, 50, and 52, wherein said nucleotide molecule encodes a protein comprising HR activity in a plant in the presence of AVR3a^{EM};
(d) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM};
(e) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions as set forth in FIG. 3;
(f) a nucleotide sequence encoding an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said nucleotide molecule encodes a protein comprising HR activity in a plant in the presence of AVR3a^{EM}, and wherein the amino acid sequence comprises at least one of the amino acid substitutions in the LRR domain as set forth in FIG. 3; and
(g) a fragment of the nucleotide sequence of any one of (a)-(f), wherein said fragment encodes a protein comprising HR activity in a plant in the presence of AVR3a^{EM}.

23. The method of claim 20, further comprising regenerating a transformed plant from said transformed cell.

24. The method of claim 20, wherein the R3a gene is native to the genome of the potato plant or was introduced into the genome of the plant or progenitor thereof by transformation.

25. The method of claim 20, wherein the R3a gene is a wild-type or mutant R3a gene.

26. A plant or plant cell produced by the method of claim 20.

27. A polypeptide comprising an amino acid sequence selected from the group consisting of:
(a) the amino acid sequence of a modified R3a protein that is capable of inducing a hypersensitive response in a plant in the presence of AVR3a^{EM};
(b) the amino acid sequence of a modified R3a protein that is capable of inducing a hypersensitive response in a plant in the presence of AVR3a^{EM} and that is capable of inducing a hypersensitive response in a plant in the presence of AVR3a^{KI};
(c) the amino acid sequence set forth in SEQ ID NO: 4, 6, 8, 10, 12, 14, 16, 18, 20, 22, 24, 26, 28, 30, 32, 34, 36, 43, 45, 47, 49, 51, or 53;
(d) an amino acid sequence encoded by the nucleotide sequence set forth in SEQ ID NO: 3, 5, 7, 9, 11, 13, 15, 17, 19, 21, 23, 25, 27, 29, 31, 33, 35, 42, 44, 46, 48, 50, or 52;
(e) an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein said polypeptide comprises HR activity in a plant in the presence of AVR3a^{EM};
(f) an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein the polypeptide comprises HR activity in a plant in the presence of AVR3a^{EM}, and wherein the amino acid sequence comprises at least one of the amino acid substitutions as set forth in FIG. 3;
(g) an amino acid sequence comprising at least 85% amino acid sequence identity to at least one amino acid sequence selected from the group consisting of SEQ ID NOS: 4, 6, 8, 10, 12, 14, 16, 18, 20, 22, 24, 26, 28, 30, 32, 34, 36, 43, 45, 47, 49, 51, and 53, wherein the polypeptide comprises HR activity in a plant in the presence of AVR3a^{EM}, and wherein the amino acid sequence comprises at least one of the amino acid substitutions in the LRR domain as set forth in FIG. 3; and
(h) a fragment of the amino acid sequence of any one of (a)-(g), wherein said polypeptide comprises HR activity in a plant in the presence of AVR3a^{EM}; and
(g) the amino acid sequence of any one of (e)-(h), wherein said polypeptide further comprises HR activity in a plant in the presence of AVR3a^{KI}.

28. A method of selecting a potato plant for enhanced resistance to Phytophthora infestans, the method comprising
(a) screening one or more potato plants or parts or cells thereof for a nucleotide sequence encoding a modified R3a protein or for a modified R3a protein, wherein the modified R3a protein is capable of inducing a hypersensitive response in a plant in the presence of AVR3a^{EM}; and
(b) selecting a potato plant comprising the nucleotide sequence encoding a modified R3a protein or the modified R3a protein.

29. The method of claim 28, wherein the modified R3a protein is selected from the group consisting of,
(i) a modified R3a protein comprising at least one amino acid substitution as set forth in FIG. 3,
(ii) a modified R3a protein comprising at least one amino acid substitution in the LRR domain as set forth in FIG. 3,
(iii) a modified R3a protein comprising an amino acid sequence that differs from the wild-type R3a amino acid sequence by a single amino acid substitution,
(iv) a modified R3a protein comprising an amino acid sequence that differs from the wild-type R3a amino acid sequence by a single amino acid substitution in the LRR domain,
(v) a modified R3a protein comprising an amino acid sequence that differs from the wild-type R3a amino acid sequence by a single amino acid substitution, wherein the single amino acid substitution is selected from the group consisting of L668P, K920E, E941K, C950R, E983K, and K1250R, and
(vi) a modified R3a protein comprising an amino acid sequence selected from the group consisting of, the amino acid sequences set forth in SEQ ID NOS: 32, 43, 45, 49, 51 and 53 and the amino acid sequences encoded by the nucleotide sequences set forth in SEQ ID NOS: 31, 42, 44, 48, 50 and 52.

30. The method of claim 28, wherein the one or more potato plants is from a population of plants that has been mutagenized or descended from plants that have been mutagenized.

31. A plant selected by the method of claim 28 or a progeny plant thereof comprising the nucleotide sequence encoding the modified R3a protein.

32. A method of enhancing the resistance of a potato plant to Phytophthora infestans, the method comprising crossing a first potato plant with a second potato plant, wherein the first potato plant was selected by the method of claim 28, wherein a progeny plant resulting from said crossing has enhanced resistance to Phytophthora infestans, when compared to the resistance of at least one of the first plant and the second plant.

33. A progeny plant produced by the method of claim 32.

34. A method for making an R protein with altered recognition specificity for an effector protein of a plant pathogen, said method comprising, substituting at least one amino acid in the amino sequence of an R protein with a different amino acid, so as to produce a modified R protein,
wherein the unmodified R protein is capable of causing a hypersensitive response when the unmodified R protein is present in a plant with a first effector protein but is not capable of causing a hypersensitive response when the unmodified R protein is present in a plant with a second effector protein, and
wherein modified R protein is capable of causing a hypersensitive response when the modified R protein is present in a plant with the second effector protein.

35. The method of claim 34, wherein the modified R protein is produced by altering the coding sequence of the R protein whereby the altered coding sequence encodes an amino acid sequence that comprises at least one amino acid substitution when compared to the amino acid sequence of the unmodified R protein.

36. The method of claim 35, wherein the coding sequence is altered by making a targeted change in one or more nucleotides in the coding sequence or by random mutagenesis.

37. The method of claim 34, further comprising testing the modified R protein to determine if it causes a hypersensitive response when the modified R protein is present in a plant with the second effector protein.

38. The method of claim 37, further comprising testing the modified R protein to determine if it causes a hypersensitive response when the modified R protein is present in a plant with the first effector protein.

39. The method of claim 37, wherein the modified R protein comprises altered recognition specificity when the modified R protein is capable of causing a hypersensitive response in a plant in the presence of the second effector protein.

40. The method of claim 34, wherein the plant pathogen is an oomycete.

41. The method of claim 40, wherein the oomycete is selected from the group consisting of Phytophthora infestans, Phytophthora sojae, Phytophthora capsici, and Phytophthora palmivora.

42. The method of claim 41, wherein the first effector is AVR3a^{KI} or AVR3a^{EM}.

43. The method of claim 34, wherein the plant is selected from the group consisting of potato, tomato, eggplant, petunia, Physalis sp., woody nightshade, garden huckleberry, gboma eggplant, Ageratum conyzoides, Solanecio biafrae, peppers, soybean, cocoa, and palms.

44. The method of claim 34, wherein the R protein is R3a.

45. A modified R protein produced by the method of claim 34.

46. A nucleic acid molecule comprising a nucleotide sequence encoding a modified R protein produced by the method of claim 34.

47. A plant or plant cell comprising in its genome the nucleic acid molecule of claim 46.

48. A method for making a modified R protein that is capable of causing in a plant a hypersensitive response of increased severity, said method comprising, substituting at least one amino acid in the amino sequence of an R protein with a different amino acid so as to produce a modified R protein, wherein the modified R protein causes a hypersensitive response in a plant in the presence of an effector protein that is of increased severity, when compared to a hypersensitive response caused in a plant by the unmodified R protein in the presence of the effector protein.

49. The method of claim 48, wherein the modified R protein is produced by altering the coding sequence of the R protein whereby the altered coding sequence encodes an amino acid sequence that comprises at least one amino acid substitution when compared to the amino acid sequence of the unmodified R protein.

50. The method of claim 48, wherein the coding sequence is altered by making a targeted change in one or more nucleotides in the coding sequence or by random mutagenesis.

51. The method of claim 48, further comprising testing the modified R protein to determine if it causes a hypersensitive response when the modified R protein is present in a plant with the effector protein.

52. The method of claim 48, wherein the effector protein is an oomycete effector protein.

53. The method of claim 52, wherein the oomycete is selected from the group consisting of Phytophthora infestans, Phytophthora sojae, Phytophthora capsici, and Phytophthora palmivora.

54. The method of claim 53, wherein the effector is AVR3a^{KI} or AVR3a^{EM}.

55. The method of claim 48, wherein the plant is selected from the group consisting of potato, tomato, eggplant, petunia, Physalis sp., woody nightshade, garden huckleberry, gboma eggplant, Ageratum conyzoides, Solanecio biafrae, peppers, soybean, cocoa, and palms.

56. The method of claim 48, wherein the R protein is R3a.

57. The method of claim 48, wherein the R protein is an R3a protein that has modified to comprise an altered recognition specificity, when compared to a wild-type potato R3a protein.

58. A modified R protein produced by the method of claim 48.

59. A nucleic acid molecule comprising a nucleotide sequence encoding a modified R protein produced by the method of claim 48.

60. A plant comprising in its genome the nucleic acid molecule of claim 59.

61. A nucleic acid molecule comprising a nucleotide sequence selected from the group consisting of:
(a) a nucleotide sequence encoding the amino acid sequence set forth in SEQ ID NO: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84;
(b) a nucleotide sequence encoding an amino acid sequence comprising at least 85% amino acid sequence identity to at least one amino acid sequence selected from the group consisting of SEQ ID NOS: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, and 84, wherein said nucleotide molecule encodes a protein comprising HR activity in a plant in the presence of at least one R protein;
(c) a fragment of the nucleotide sequence of (a) or (b), wherein said fragment encodes a protein comprising HR activity in a plant in the presence of at least one R protein; and
(d) a nucleotide sequence that is fully complementary to the nucleotide sequence of any one of (a)-(c).

62. An expression cassette comprising the nucleic acid molecule of claim 61 operably linked to a promoter.

63. A non-human host cell comprising the expression cassette of claim 62.

64. A polypeptide comprising an amino acid sequence selected from the group consisting of:
(a) the amino acid sequence set forth in SEQ ID NO: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84;
(b) an amino acid sequence comprising at least 85% amino acid sequence identity to at least one amino acid sequence selected from the group consisting of SEQ ID NOS: 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, and 84, wherein said polypeptide comprises HR activity in a plant in the presence of at least one R protein; and
(c) a fragment of the amino acid sequence of (a) or (b), wherein said polypeptide comprises HR activity in a plant in the presence of at least one R protein.
